(19) United States
(12) Patent Application Publication
(10) Pub. No.: US 2010/0211114 A1
(43) Pub. Date: Aug. 19, 2010

(54) Polyaxial bone anchor with shelf capture connection

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 12/799,290

(22) Filed: Apr. 21, 2010

Related U.S. Application Data
(63) Continuation-in-part of application No. 12/584,981, filed on Sep. 15, 2009.
Application No. 12/584,981 is a continuation-in-part of application No. 12/587,244, filed on Oct. 2, 2009.
Application No. 12/587,244 is a continuation of application No. 10/818,554, filed on Apr. 5, 2004, now Pat. No. 7,662,175.
Application No. 10/818,554 is a continuation-in-part of application No. 10/651,003, filed on Aug. 28, 2003.
Application No. 10/651,003 is a continuation of application No. 10/464,633, filed on Jun. 18, 2003, now Pat. No. 6,716,214.
Application No. 10/464,633 is a continuation-in-part of application No. 12/154,460, filed on May 23, 2008.
Application No. 12/154,460 is a continuation-in-part of application No. 11/140,343, filed on May 27, 2005.
Application No. 11/140,343 is a continuation-in-part of application No. 10/818,555, filed on Apr. 5, 2004.
Application No. 10/818,555 is a continuation of application No. 10/464,633, filed on Jun. 18, 2003, now Pat. No. 6,716,214.
Application No. 10/464,633 is a continuation-in-part of application No. 10/651,003, filed on Aug. 28, 2003.
Application No. 10/651,003 is a continuation-in-part of application No. 12/290,244, filed on Oct. 29, 2008.
Application No. 12/290,244 is a continuation-in-part of application No. 11/522,503, filed on Sep. 14, 2006.
Application No. 11/522,503 is a continuation-in-part of application No. 11/024,543, filed on Dec. 20, 2004, now Pat. No. 7,204,838.
(60) Provisional application No. 61/214,447, filed on Apr. 23, 2009.
(60) Provisional application No. 61/214,872, filed on Apr. 29, 2009.
(60) Provisional application No. 61/201,806, filed on Dec. 15, 2008.
(60) Provisional application No. 60/931,362, filed on May 23, 2007.
(60) Provisional application No. 61/000,964, filed on Oct. 30, 2007.

(51) Int. Cl. A61B 17/86

(52) U.S. Cl. 606/302; 606/305; 606/304

(57) ABSTRACT

A medical implant assembly includes a polyaxial bone anchor having a shank, a receiver and a retainer structure for capturing and supporting an upper end portion of the shank in the receiver. The shank includes radially extending projections on its upper end and the retainer structure includes shelves with seating surfaces for receiving and supporting the shank projections. Some embodiments include a lower compression insert.

### CROSS REFERENCE TO RELATED APPLICATIONS

This application claims the benefit of U.S. Provisional Application No. 61/214,447, filed Apr. 23, 2009 and U.S. Provisional Application No. 61/214,872, filed Apr. 29, 2009 both of which are incorporated by reference herein. This application is also a continuation-in-part of U.S. patent application Ser. No. 12/584,981 filed Sep. 15, 2009 that claims the benefit of U.S. Provisional Application No. 61/201,806 filed Dec. 15, 2008, both of which are incorporated by reference herein.

This application is also a continuation-in-part of U.S. patent application Ser. No. 12/587,244 filed Oct. 2, 2009 that is a continuation of U.S. patent application Ser. No. 10/818,554, filed Apr. 5, 2004, now U.S. Pat. No. 7,662,175, that is both a continuation-in-part of U.S. patent application Ser. No. 10/651,003 filed Aug. 28, 2003 and a continuation of U.S. patent application Ser. No. 10/464,633 filed Jun. 18, 2003, now U.S. Pat. No. 6,716,214, all of which are incorporated herein by reference herein.

This application is also a continuation-in-part of U.S. patent application Ser. No. 12/154,460 filed May 23, 2008 that claims the benefit of U.S. Provisional Application No. 60/931,362 filed May 23, 2007, and is a continuation-in-part of U.S. patent application Ser. No. 11/140,343 filed May 27, 2005, all of which are incorporated by reference herein. The Ser. No. 12/154,460 application is also a continuation-in-part of U.S. patent application Ser. No. 10/818,555 filed Apr. 5, 2004 that is a continuation of U.S. patent application Ser. No. 10/464,633 filed Jun. 18, 2003, now U.S. Pat. No. 6,716,214 and a continuation-in-part of U.S. patent application Ser. No. 10/651,003, filed Aug. 28, 2003, all of which are incorporated by reference herein.

This application is also a continuation-in-part of U.S. patent application Ser. No. 12/290,244 filed Oct. 29, 2008 that claims the benefit of U.S. Provisional Application Ser. No. 61/000,964 filed Oct. 30, 2007 and that is a continuation-in-part of U.S. patent application Ser. No. 11/522,503 filed Sep. 14, 2006 that is a continuation-in-part of U.S. patent application Ser. No. 11/024,543 filed Dec. 20, 2004, now U.S. Pat. No. 7,204,838, all of which are incorporated by reference herein.

### BACKGROUND OF THE INVENTION

Many spinal surgery procedures require securing various implants to bone and especially to vertebrae along the spine. For example, longitudinal connecting members such as elongate rods are often required that extend along the spine to provide support to vertebrae that have been damaged or weakened due to injury, disease or the like. Such rods must be supported by certain vertebra and support other vertebra. The most common mechanism for providing such structure is to implant bone screws into certain bones which then in turn support the rod or are supported by the rod. Bone screws of this type may have a fixed head or rod receiver relative to a shank thereof. In the fixed bone screws, the receiver cannot be moved relative to the shank and the rod or other longitudinal connecting member must be favorably positioned in order for it to be placed within the receiver. This is sometimes very difficult or impossible to do so polyaxial bone screws are commonly used. Polyaxial bone screws allow rotation of the head or receiver about the shank until a desired rotational position is achieved for the receiver relative to the shank after which the longitudinal connecting member can be inserted and the position of the receiver eventually locked with respect to movement relative to the shank.

The present invention is directed to such swivel head type bone screws and, in particular, to swivel head bone screws having an open head or receiver that allows placement of the longitudinal connecting member within the receiver and then subsequent closure by use of a closure top, plug or the like to capture the connector in the receiver of the screw.

### SUMMARY OF THE INVENTION

A polyaxial bone screw assembly of the present invention includes a shank having a generally elongate body with an upper end portion and a lower threaded portion for fixation to a bone. The bone screw assembly further includes a receiver having a top portion and a base. The top portion is open and has a channel. The base includes an inner seating surface partially defining a cavity and has a lower aperture or opening. The channel of the top portion of the receiver communicates with the cavity, which in turn communicates with an opening to an exterior of the base of the receiver. The shank upper portion is disposed in the receiver cavity and the shank extends through the receiver base opening. A shank capture connection is provided by the shank upper portion having at least one projection cooperating with a retainer structure that includes at least one generally vertical passageway for the projection and at least one shelf structure that engages and supports the at least one projection, the retainer structure configured for polyaxial motion with respect to the receiver. The shelf may be located midway, near a top or anywhere along a height of the retainer. The retainer can be integral or have a slit or slot. The shank projection may be press fit against the retainer structure or portions of the shank or portions of the retainer structure may be crimped, cut or otherwise deformed so as to be pressed against one another to lock and hold the parts together. The shank and retainer can also be held together by spot or laser welding. In some embodiments of the invention, the shank has an upper surface extending above the captured retainer that exclusively engages a compression or pressure insert that in turn engages a longitudinal connecting member being supported within the receiver. In such embodiments, the shank and retainer can alternatively be held together by blocking wedges that fill the passageway. In other embodiments, the shank upper surface directly engages the longitudinal connecting member and the shank and retainer are held together, as described above. In addition, pinning methods can be used to hold together the shank and retainer, the shank projection or projections thereby being supported by the receiver shelf or shelves.

Objects of the invention include providing apparatus and methods that are easy to use and especially adapted for the intended use thereof and wherein the apparatus are comparatively inexpensive to make and suitable for use. Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged and exploded perspective view of a polyaxial bone screw assembly according to the invention including a bone screw shank, a receiver, a retainer structure and further shown with a longitudinal connecting member in the form of a rod and a closure structure having a break-off head.

FIG. 2 is an enlarged perspective view of the retainer structure of FIG. 1.

FIG. 3 is an enlarged front elevational view of the retainer structure of FIG. 1.

FIG. 5 is an enlarged bottom plan view of the retainer structure of FIG. 1.

FIG. 6 is an enlarged front elevational view of the receiver and retainer structure of FIG. 1 with portions broken away to show the detail thereof, showing the retainer structure in a first stage of assembly.

FIG. 7 is an enlarged front elevational view, simile to FIG. 6 with the retainer structure shown in a subsequent stage of assembly.

FIG. 8 is an enlarged and partial front elevational and partially exploded view of the receiver, retainer structure and bone screw shank of FIG. 1 shown in a stage of assembly subsequent to that shown in FIG. 7.

FIG. 9 is an enlarged and partial and partially exploded bottom perspective view of the stage of assembly shown in FIG. 8.

FIG. 10 is an enlarged and partial top plan view of the shank, retainer structure and receiver of FIG. 1 shown in a stage of assembly subsequent to that shown in FIG. 9.

FIG. 11 is an enlarged and partial top plan view, similar to FIG. 10, showing a further stage of assembly of the shank with the retainer structure.

FIG. 12 is an enlarged and partial front elevational view of the shank, retainer structure and receiver of FIG. 1 in the stage of assembly shown in FIG. 10, with portions broken away to show the detail thereof and with portions of the retainer structure shown in phantom.

FIG. 13 is an enlarged and partial front elevational view of the shank, retainer structure and receiver of FIG. 1 in the stage of assembly shown in FIG. 11, with portions broken away to show the detail thereof and with portions of the retainer structure shown in phantom.

FIG. 14 is an enlarged and partial front elevational view of the entire assembly of FIG. 1 with portions broken away to show the detail thereof, with portions of the retainer structure and the shank shown in phantom, and the assembly shown in a fully assembled stage with the break-off head of the closure removed.

FIG. 15 is an enlarged and exploded perspective view of a second embodiment of a polyaxial bone screw assembly according to the invention including a bone screw shank, a receiver, a retainer structure and further shown with a longitudinal connecting member in the form of a rod and a closure structure having a break-off head.

FIG. 16 is an enlarged perspective view of the retainer structure of FIG. 15.

FIG. 17 is an enlarged front elevational view of the retainer structure of FIG. 15 with portions shown in phantom.

FIG. 18 is an enlarged top plan view of the retainer structure of FIG. 15 with portions shown in phantom.

FIG. 19 is an enlarged bottom plan view of the retainer structure of FIG. 15 with portions shown in phantom.

FIG. 20 is an enlarged front elevational view of the receiver and retainer structure of FIG. 15 with portions broken away to show the detail thereof, showing the retainer structure in a first stage of assembly in phantom and a second stage of assembly in solid lines.

FIG. 21 is an enlarged and partial front elevational view of the receiver, retainer structure and bone screw shank of FIG. 15 with portions broken away to show the detail thereof and shown in a stage of assembly subsequent to that shown in FIG. 20.

FIG. 22 is an enlarged and partial front elevational view of the shank, retainer structure and receiver of FIG. 15 with portions broken away to show the detail thereof and shown in a stage of assembly subsequent to that shown in FIG. 21.

FIG. 23 is an enlarged and partial front elevational view of the shank, retainer structure and receiver of FIG. 15 with portions broken away to show the detail thereof and shown in a stage of assembly subsequent to that shown in FIG. 22 with a crimping tool pressing the retainer structure into crimped engagement with the shank.

FIG. 24 is an enlarged and partial front elevational view of the shank, retainer structure and receiver of FIG. 15 with portions broken away to show the detail thereof and shown in a stage subsequent to that shown in FIG. 23 with the crimping tool being moved away from the assembly.

FIG. 25 is a top plan view of the shank, retainer structure and receiver of FIG. 15 with portions broken away to show the detail thereof and shown in a stage subsequent to that shown in FIG. 24, with the retainer structure being crimped into engagement with the shank at four locations.

FIG. 26 is an enlarged and exploded perspective view of a third embodiment of a polyaxial bone screw assembly according to the invention including a bone screw shank, a receiver, a retainer structure and further shown with a longitudinal connecting member in the form of a rod and a closure structure having a break-off head.

FIG. 27 is an enlarged perspective view of the retainer structure of FIG. 26.

FIG. 28 is an enlarged front elevational view of the retainer structure of FIG. 26 with portions shown in phantom.

FIG. 29 is an enlarged top plan view of the retainer structure of FIG. 26 with portions shown in phantom.

FIG. 30 is an enlarged bottom plan view of the retainer structure of FIG. 26 with portions shown in phantom.

FIG. 31 is an enlarged and partial front elevational view of the receiver, retainer structure and shank of FIG. 26 with portions broken away to show the detail thereof and portions in phantom, showing an interim stage of assembly of the shank and the retainer structure with a pair of pins.

FIG. 32 is an enlarged and partial front elevational view with portions broken away, similar to FIG. 31, showing the pair of pins fully inserted into and engaged with the shank and retainer structure.

FIG. 33 is an enlarged and exploded perspective view of a fourth embodiment of a polyaxial bone screw assembly according to the invention including a bone screw shank, a receiver, a retainer structure and further shown with a longitudinal connecting member in the form of a rod and a closure structure having a break-off head.

FIG. 34 is an enlarged perspective view of the retainer structure of FIG. 33.

FIG. 35 is an enlarged front elevational view of the retainer structure of FIG. 33.

FIG. 36 is an enlarged top plan view of the retainer structure of FIG. 33.

FIG. 37 is an enlarged bottom plan view of the retainer structure of FIG. 33.

FIG. 38 is a cross-sectional view taken along the line 38-38 of FIG. 36.

FIG. 39 is an enlarged and partial perspective view of the shank of FIG. 33.

FIG. 40 is an enlarged and partial top plan view of the shank and retainer structure of FIG. 33 shown in a stage of assembly.

FIG. 41 is an enlarged and partial top plan view of the shank and retainer structure of FIG. 33, shown in a stage of assembly subsequent to that shown in FIG. 40.

FIG. 42 is an enlarged and partial top plan view of the shank and retainer structure of FIG. 33, shown in a stage of assembly subsequent to that shown in FIG. 41.

FIG. 43 is a partial cross-sectional view taken along the line 43-43 of FIG. 42.

FIG. 44 is an enlarged and exploded perspective view of a fifth embodiment of a polyaxial bone screw assembly according to the invention including a bone screw shank, a receiver, a retainer structure, a lower pressure insert and further shown with a longitudinal connecting member in the form of a rod and a closure structure.

FIG. 45 is an enlarged perspective view of the retainer structure of FIG. 44.

FIG. 46 is an enlarged top plan view of the retainer structure of FIG. 44.

FIG. 47 is an enlarged top plan view of the shank of FIG. 44.

FIG. 48 is an enlarged top plan view of the pressure insert of FIG. 44.

FIG. 49 is an enlarged bottom plan view of the pressure insert of FIG. 44.

FIG. 50 is a cross-sectional view taken along the line 50-50 of FIG. 48.

FIG. 51 is an enlarged and partial perspective view of the shank and attached retainer structure of FIG. 44.

FIG. 52 is an enlarged and partial front elevational view of the assembly of FIG. 44 with portions broken away to show the detail thereof.

FIG. 53 is an enlarged perspective view of an alternative retainer structure according to the invention.

FIG. 54 is a reduced and partial perspective view of the retainer structure of FIG. 53 shown attached to a shank identical or similar to the shank of FIG. 44.

FIG. 55 is an enlarged perspective view of another alternative retainer structure according to the invention.

FIG. 56 is a reduced and partial perspective view of the retainer structure of FIG. 55 shown in the process of attachment to a shank identical or similar to the shank of FIG. 44.

FIG. 57 is an enlarged and partial perspective view, similar to FIG. 56, showing the retainer structure attached to the shank and with portions shown in phantom.

FIG. 58 is an enlarged and partial front elevational view of an alternative polyaxial bone screw according to the invention shown with the shank and retainer of FIGS. 55-57, with portions broken away to show the detail thereof.

FIG. 59 is an enlarged perspective view of an alternative retainer structure for use in a polyaxial bone screw according to the invention.

FIG. 60 is an enlarged perspective view of another alternative retainer structure for use in a polyaxial bone screw according to the invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of bone attachment assemblies of the application and cooperating connecting members in actual use.

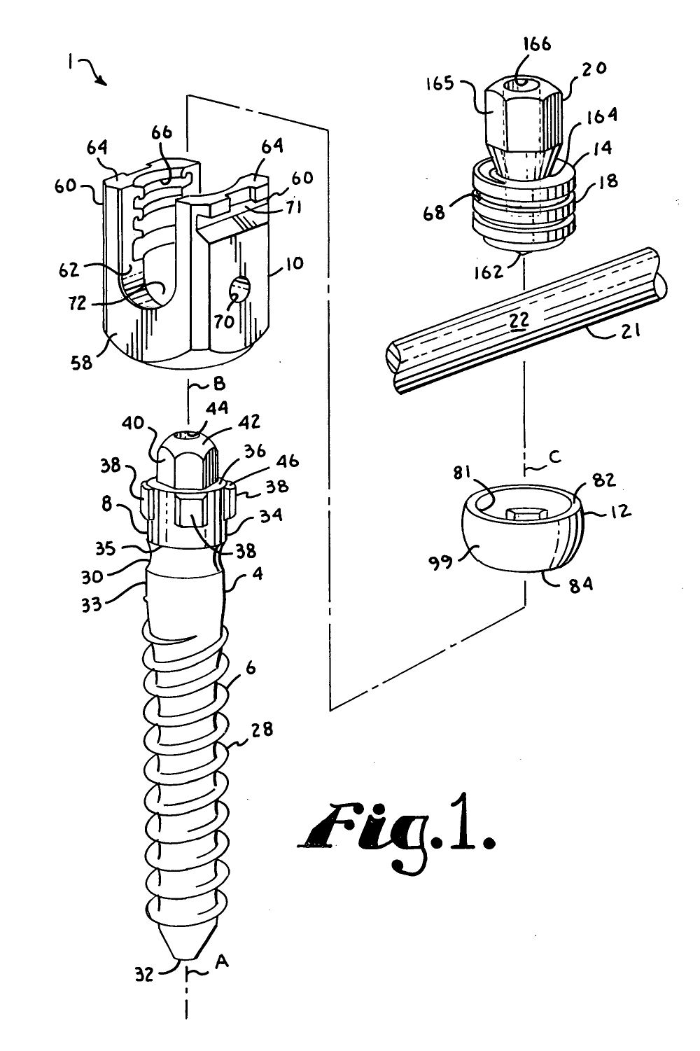

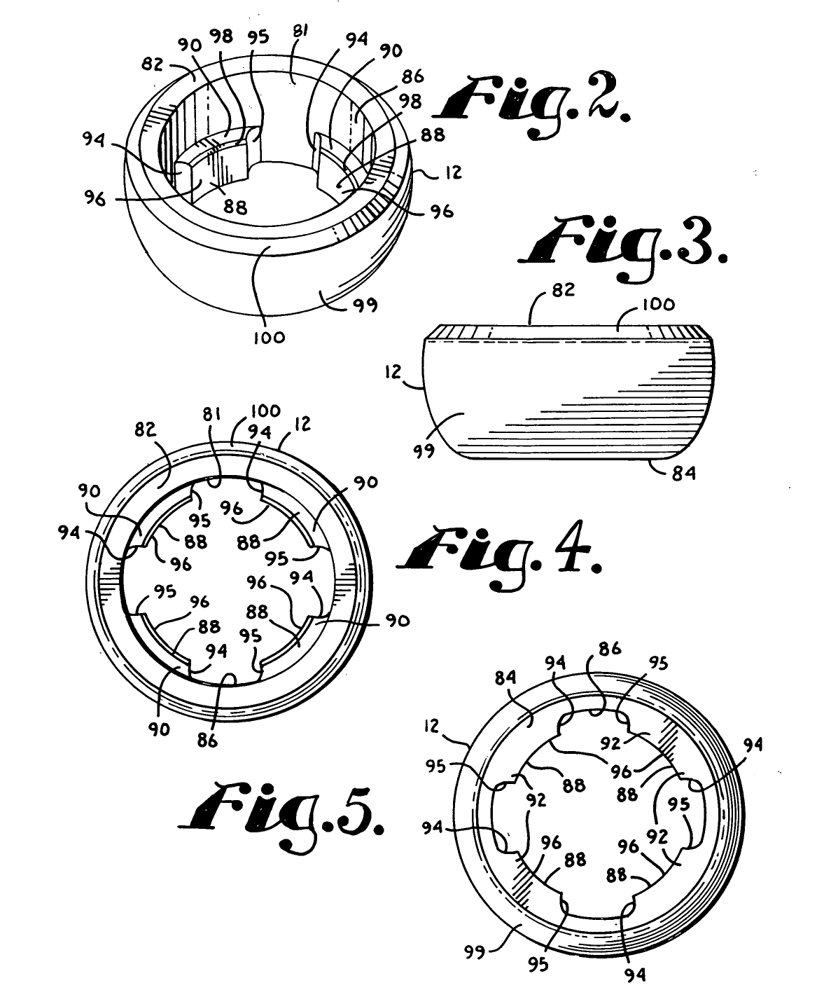

With reference to FIGS. 1-14, the reference number 1 generally represents an embodiment of a polyaxial bone screw apparatus or assembly according to the present invention. The assembly 1 includes a shank 4 that further includes a threaded body 6 integral with an upper portion 8; a receiver 10; a retainer structure 12 fixable to the upper portion 8 and swivelable with respect to the receiver 10; and a closure structure 14. The shank 4, receiver 10, and retainer structure 12 are typically factory assembled prior to implantation of the shank body 6 into a vertebra (not shown).

With further reference to FIG. 1, the closure structure 14 further includes a lower fastener portion 18 and a break-off head or top 20. The fastener portion 18 engages a longitudinal connecting member such as a rod 21, for example, having a cylindrical surface 22 shown in FIGS. 1 and 14. The fastener portion 18 presses against the rod 21 that in turn presses upon the shank upper portion 8 which biases the retainer structure 12 into fixed frictional contact with the receiver 10, so as to fix the rod 21 relative to the vertebra (not shown). The receiver 10 and the shank 4 cooperate in such a manner that the receiver 10 and the shank 4 can be secured at any of a plurality of angles, articulations or rotational alignments relative to one another and within a selected range of angles both from side to side and from front to rear, to enable flexible or articulated engagement of the receiver 10 with respect to the shank 4 until both are locked or fixed relative to each other near the end of an implantation procedure.

With particular reference to FIGS. 1 and 8-14, the shank 4 is elongate, with the shank body 6 having a helically wound bone implantable thread 28 extending from near a neck 30 located adjacent to the upper portion 8 to a tip 32 of the body 6 and extending radially outwardly therefrom. During use, the body 6 utilizing the thread 28 for gripping and advancement is implanted into the vertebra (not shown) leading with the tip 32 and driven down into the vertebra with an installation or driving tool, so as to be implanted in the vertebra to near the neck 30, and as is described more fully in the paragraphs below. The shank 4 has an elongate axis of rotation generally identified by the reference letter A.

The neck 30 extends axially upwardly and away from the shank body 6. The neck 30 is of slightly reduced radius as compared to an adjacent top 33 of the threaded body 6. Further extending axially upwardly and away from the neck 30 is the shank upper portion 8 that provides a connective or capture apparatus disposed at a distance from the threaded body top 33 and thus at a distance from the vertebra when the body 6 is implanted in the vertebra.

The shank upper portion 8 is configured for a fixed connection between the shank 4 and the retainer structure 12 and a pivotable connection between the shank 4/retainer structure 12 combination and the receiver 10 prior to fixing of the shank in a desired position with respect to the receiver 10. The upper portion 8 generally includes a substantially cylindrical body 34 having a lower rim 35 located adjacent the neck 30 and an opposed upper annular surface 36. In the illustrated embodiment, four evenly spaced projections 38 extend radially from the body 34 and are located near the upper surface 36. Other embodiments of the invention include at least one and up to a plurality of projections 38. An external tool engagement drive feature or structure 40 extends upwardly and axially from the upper surface 36 and is illustrated as a multi-faceted hex-shape structure sized and shaped to mate with a socket driving tool (not shown) having an internal drive configured to fit about the tool engagement structure 40 for both driving and rotating the shank body 6 into the vertebra. A top surface 42 of the drive structure 40 is preferably curved, radiused or domed shaped as shown in the drawings, for contact and positive mating engagement with the surface 22 of the rod 21 when the bone screw assembly 1 is fully assembled, as shown in FIG. 14 and in any pivotal alignment of the shank 4 relative to the receiver 10. In the illustrated embodiment, the surface 42 is smooth. While not required in accordance with the practice of the invention, the surface 42 may be scored or knurled to further increase frictional positive mating engagement between the surface 42 and the rod 21. The shank 4 shown in the drawings is cannulated, having a small central bore 44 extending an entire length of the shank 4 along the axis A. The bore 44 is defined by an inner cylindrical wall of the shank 4 and has a circular opening at the shank tip 32 and an upper opening communicating with the external drive top surface 42. The bore 44 is coaxial with the threaded body 6 and the upper portion 8. The bore 44 provides a passage through the shank 4 interior for a length of wire (not shown) inserted into the vertebra (not shown) prior to the insertion of the shank body 6, the wire providing a guide for insertion of the shank body 6 into the vertebra (not shown).

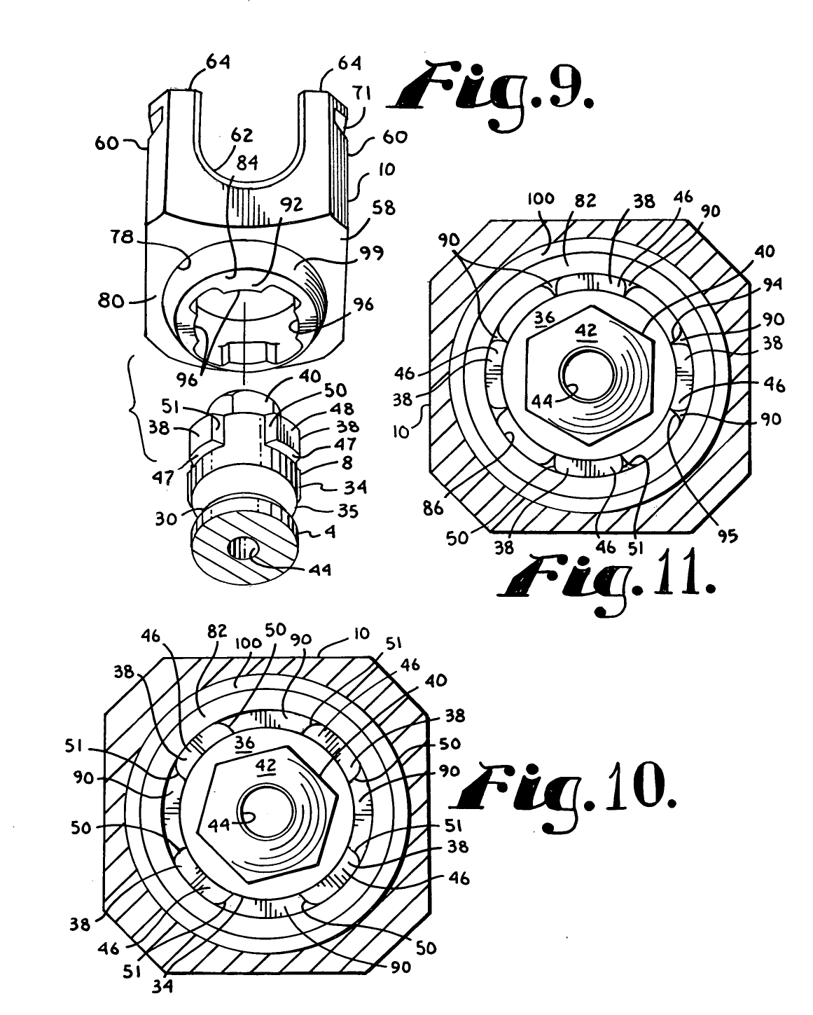

In the illustrated embodiment, each of the four similarly sized and shaped spaced projections 38 include a planar top surface 46 and an opposed substantially planar bottom surface 47, an outer substantially cylindrical surface 48 and a pair of opposed, curved side surfaces 50 and 51. Each of the surfaces 46, 47, 50 and 51 terminate at the cylindrical surface 48 at one side thereof and at the cylindrical body 34 at the other side thereof. The side surfaces 50 and 51 and the surface 48 run substantially parallel to the axis A. Each top surface 46 slopes downwardly (toward the shank body 6) in a direction from the cylindrical body 34 to the cylindrical surface 48. Each bottom surface 47 slopes upwardly (toward the drive structure 40) in a direction from the cylindrical body 34 to the cylindrical surface 48. As best illustrated in FIGS. 10 and 11, the opposed curved side surfaces 50 and 51 are each convex and similarly shaped.

To provide a biologically active interface with the bone, the threaded shank body 6 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

Referring to FIGS. 1 and 6-14, the receiver 10 has a generally squared-off U-shaped appearance with a partially cylindrical inner profile and a substantially faceted outer profile; however, the outer profile could also be of another configuration, for example, curved or cylindrical. A receiver axis of rotation B, as shown in FIG. 1, is aligned with the axis of rotation A of the shank 4 during assembly of the receiver 10 with the shank 4 and the retainer structure 12. After the receiver 10 is pivotally connected to the shank 4, and the assembly 1 is implanted in a vertebra (not shown), the axis B is typically disposed at an angle with respect to the axis A of the shank 4.

The receiver 10 includes a base 58 and pair of spaced and generally parallel arms 60 that form an open generally U-shaped channel 62 therebetween that is open at distal ends 64 of such arms. The receiver arms 60 each include radially inward or interior surfaces that have a discontinuous guide and advancement structure 66 mateable with cooperating structure 68 on the fastening portion 18 of the closure structure 14. The guide and advancement structure 66 may be a partial helically wound flangeform configured to mate under rotation with a similar structure on the closure structure 14 or a buttress thread, a square thread, a reverse angle thread or other thread like or non-thread like helically wound advancement structure for operably guiding under rotation and advancing the closure structure 14 downward between the receiver arms and having such a nature as to resist splaying of the receiver arms 60 when the closure structure 14 is advanced there-between. The illustrated receiver arms 60 include opposed tool engaging apertures 70 formed on or through outer surfaces of such arms as well as opposed tool engaging grooves 71. The apertures 70 and/or grooves 71 may be used for holding the receiver 10 during assembly with the shank 4 and the retainer structure 12, during the implantation of the shank body 6 into a vertebra (not shown) and assembly with the rod 21 and the closure structure 14. It is foreseen that tool receiving grooves or apertures may be configured in a variety of shapes and sizes and be disposed at other locations on the receiver arms 60.

With further reference to FIGS. 1 and 6-14, communicating with and located beneath the U-shaped channel 62 of the receiver 10 at the base portion 58 thereof is a chamber or cavity, generally 72, defined in part by an inner substantially cylindrical surface 74 and a substantially spherical seating surface portion 76. The cylindrical surface 74 that defines a portion of the cavity 72 opens upwardly into the channel 62. The inner surface 76 that is located below the surface 74 is sized and shaped for mating with the retainer structure 12 as will be described in greater detail below. The surface portion 76 communicates with a lower opening 78 that communicates with both the cavity 72 and a receiver lower exterior or bottom 80 of the base 58. The opening 78 is substantially coaxially aligned with respect to the rotational axis B of the receiver 10. The opening 78 is also sized and shaped to be smaller than an outer radial dimension of the retainer structure 12, so as to form a restriction to prevent the structure 12 and attached shank portion 8 from passing through the cavity 72 and out the lower exterior 80 of the receiver 10 during operation thereof.

The retainer structure or retainer 12 is used to capture the shank upper portion 8 and retain the upper portion 8 within the receiver 10 as well as swivel or articulate with respect to the receiver 10. The retainer 12, best illustrated in FIGS. 1-5 has an operational central axis that is the same as the rotational axis A associated with the shank 4, but when the retainer 12 is separated from the shank 4, the axis of rotation is identified as axis C, as shown in FIG. 1. The retainer 12 has a central bore 81 that passes entirely through the retainer 12 from a top surface 82 to a bottom surface 84 thereof. The bottom surface 84 is substantially planar and disposed perpendicular to the axis C. A first inner cylindrical surface 86 defines a substantial portion of the bore 81. The cylindrical surface 86 is sized and shaped to be slidingly received about the cylindrical body portion 34 of the shank upper portion 8. Extending inwardly radially from the surface 86 toward the axis C are four evenly spaced shelves 88 sized and shaped to cooperate with the projections 38 of the shank upper portion 8 for fixing the retainer 12 to the shank upper portion 8. The shelves 88 extend from at or near the retainer bottom 84 to a location spaced from the retainer top 82. The shelves 88 are sized and shaped to provide direct mating support with each projection 38 of the shank upper portion 8. The shelves 88 are also spaced from the top surface 82 to provide adequate space along the surface 81 for loading rotation and placement of the projections 38 of the shank upper portion 8 with respect to the retainer 12 during assembly within the receiver 10 of the bone screw 1. Each of the illustrated shelves 88 includes an upper seating surface 90 and an opposed bottom surface 92 that is flush and integral with the bottom surface 84 of the retainer 12. The illustrated seating surfaces 90 are each disposed about midway between the top 82 and the bottom 84 of the retainer 12, but may be located slightly higher or lower along the surface 86. Each shelf 88 further includes opposed side surfaces 94 and 95 running from the bottom surface 92 to the seating surface 90. Each of the surfaces 94 and 95 are curved and substantially concave. Each shelf includes an inner cylindrical surface 96 sized and shaped to slidingly mate with the surfaces 48 of the projections 38 of the shank upper portion 8. The seating surfaces 90 are sized to receive the projections 38 at the surfaces 47 thereof. The surfaces 90 may be slightly sloped for receiving the surfaces 47. The shelves 88 are also slightly wider than the projections 38 at the side surfaces 94 and 95 so as to advantageously accommodate a spot weld or other fixing or adhering structure or substance at the ends of the seating surface 90 once each of the surfaces 47 engage one of the seating surfaces 90 as shown in FIG. 14. The illustrates shelves 88 also each include a beveled surface 98 disposed between the seating surface 90 and the inner cylindrical surface 96 and running from the side surface 94 to the side surface 95.

The retainer 12 also has a radially outer partially spherically shaped surface 99 sized and shaped to mate with the partial spherical shaped seating surface 76 of the receiver 10. The surface 99 includes an outer radius that is larger than a radius of the lower opening 78 of the receiver 10, thereby prohibiting the retainer 12 and the shank upper portion 8 from passing through the opening 78 once the retainer 12 is fixed to the shank upper portion 8 within the receiver cavity 72. Although not required, it is foreseen that the outer partially spherically shaped surface 99 may be a high friction surface such as a knurled surface or the like. The illustrated retainer 12 further includes a beveled surface 100 located between the top surface 82 and the curved outer surface 99.

The longitudinal connecting member 21 that is utilized with the assembly 1 can be any of a variety of implants utilized in reconstructive spinal surgery, and is illustrated as a cylindrical elongate structure or rod having the cylindrical surface 22 of uniform diameter and having a generally smooth surface. The longitudinal connecting member 21 may be made from metal, metal alloys or other suitable materials, including plastic polymers such as polyetheretherketone (PEEK), ultra-high-molecular weight-polyethylene (UHMWP), polyurethanes and composites. The illustrated longitudinal connecting member 21 is preferably sized and shaped to snugly seat near the bottom of the U-shaped channel 62 of the receiver 10 and, during normal operation, is positioned slightly above the bottom of the channel 62. In particular, the longitudinal connecting member 21 normally directly or abutingly engages the shank top surface 42 and is biased against the dome shank top surface 42, consequently biasing the shank 4 downwardly in a direction toward the base 58 of the receiver 10 when the assembly 1 is fully assembled. For this to occur, the shank top surface 42 must extend at least slightly into the space of the channel 62 when the retainer structure 12 is snugly seated against the receiver seating surface 76. The shank 4 and the retainer 12 are thereby locked or held in position relative to the receiver 10 by the longitudinal connecting member 21 firmly pushing downward on the shank top surface 42 as illustrated, for example, in FIG. 14.

Longitudinal connecting members may take a variety of shapes, including but not limited to rods or bars of oval, rectangular or other curved or polygonal cross-section. Furthermore, the connector 21 may be a component of a longer overall dynamic stabilization connecting member, with cylindrical or bar-shaped portions sized and shaped for being received by the receiver 10 that may have a U- or rectangular shaped channel for closely receiving the longitudinal connecting member. The connector 21 may be integral or otherwise fixed to a bendable or damping component that is sized and shaped to be located between adjacent pairs of bone screw assemblies 1, for example. Such a rod or bar component may be made from a variety of materials including metal, metal alloys or other suitable materials, including, but not limited to plastic polymers such as polyetheretherketone (PEEK), ultra-high-molecular weight-polyethylene (UHMWP), polyurethanes and composites, including composites containing carbon fiber, as well as resorbable materials, such as polylactic acids.

With reference to FIGS. 1 and 14, the closure structure or closure top 14 can be any of a variety of different types of closure structures for use in conjunction with the present invention with suitable mating structure on the upstanding arms 60. In the embodiment shown, the closure top 14 fastener portion 18 is rotatably received between the spaced arms 60. The fastener 18 includes the helically wound guide and advancement structure 68 that is sized, shaped and positioned so as to engage and interlock with the guide and advancement structure 66 on the arms 60 to provide for rotating advancement of the fastener 18 into the receiver 10 when rotated clockwise and, in particular, to cover the upwardly open portion of the U-shaped channel 62 near the arm ends 64 to capture the longitudinal connecting member 21 without splaying of the arms 60. The guide and advancement structure 68 utilized in accordance with the present invention may take a variety of forms, including the illustrated substantially square thread and also those described in Applicant's U.S. Pat. No. 6,726,689, which is incorporated herein by reference.

The fastener 18 includes a base surface 160 that is illustrated as having a projection or point 162 for engaging and/or penetrating the surface 22 of the longitudinal connecting member 21. The fastener 18 operably biases against the longitudinal connecting member 21 by advancement and applies pressure to the longitudinal connecting member 21 under torquing, so that the longitudinal connecting member 21 is urged downwardly against the shank top end surface 42 that extends up into the channel 62. In the illustrated embodiment, downward biasing of the shank top surface 42 operably produces a frictional engagement between the longitudinal connecting member 21 and surface 42 and also urges the retainer structure 12 toward the base 58 of the receiver 10, so as to frictionally seat the retainer structure external spherical surface 99 fixedly against the partial internal spherical seating surface 76 of the receiver 10, also fixing the shank 4 and retainer structure 12 in a selected, rigid position relative to the receiver 10.

In the embodiment shown, the closure structure break-off head 20 is secured to the fastener portion 18 at a neck 164 that is sized and shaped so as to break away at a preselected torque that is designed to properly seat the retainer 12 in the receiver 10. The break-off head 20 includes an external faceted surface 165 that is sized and shaped to receive a conventional mating socket type head of a driving tool (not shown) to rotate and torque the closure structure 14. The break-off head 20 also includes a central bore 166 or other drive or manipulation apertures for operably receiving manipulating tools. The closure structure 14 also includes removal tool engagement structure which in the present embodiment is illustrated as a hex-shaped aperture 168 that is axially aligned with and disposed in the fastener portion 18. The aperture 168 is accessible after the break-off head 20 breaks away from the fastener portion 18. The aperture 168 is coaxial with the helically wound guide and advancement structure 68 and is designed to receive a driving tool, such as a hex tool of an Allen wrench type, into the aperture 168 for rotating the closure structure fastener portion 18 subsequent to installation so as to provide for removal thereof, if necessary. The aperture 168 may take a variety of tool-engaging forms and may include one or more apertures of various shapes, such as a pair of spaced apart apertures, or a left hand threaded bore, or an easy-out engageable step down bore, or a Torx aperture, or a multi-lobular aperture or the like.

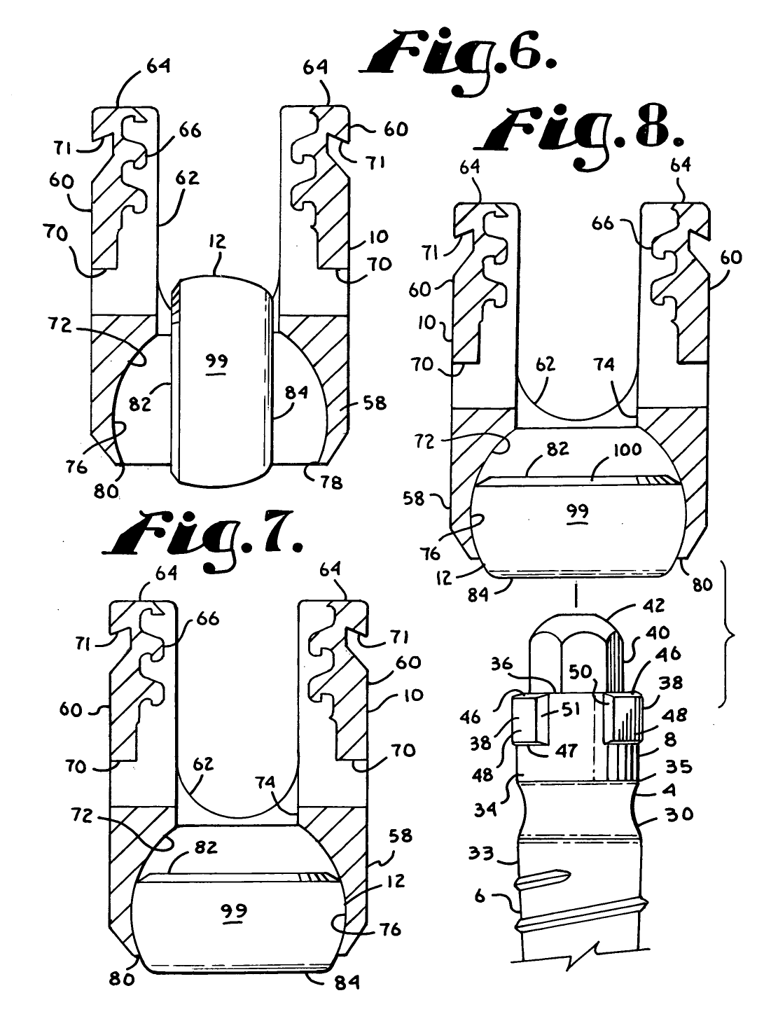

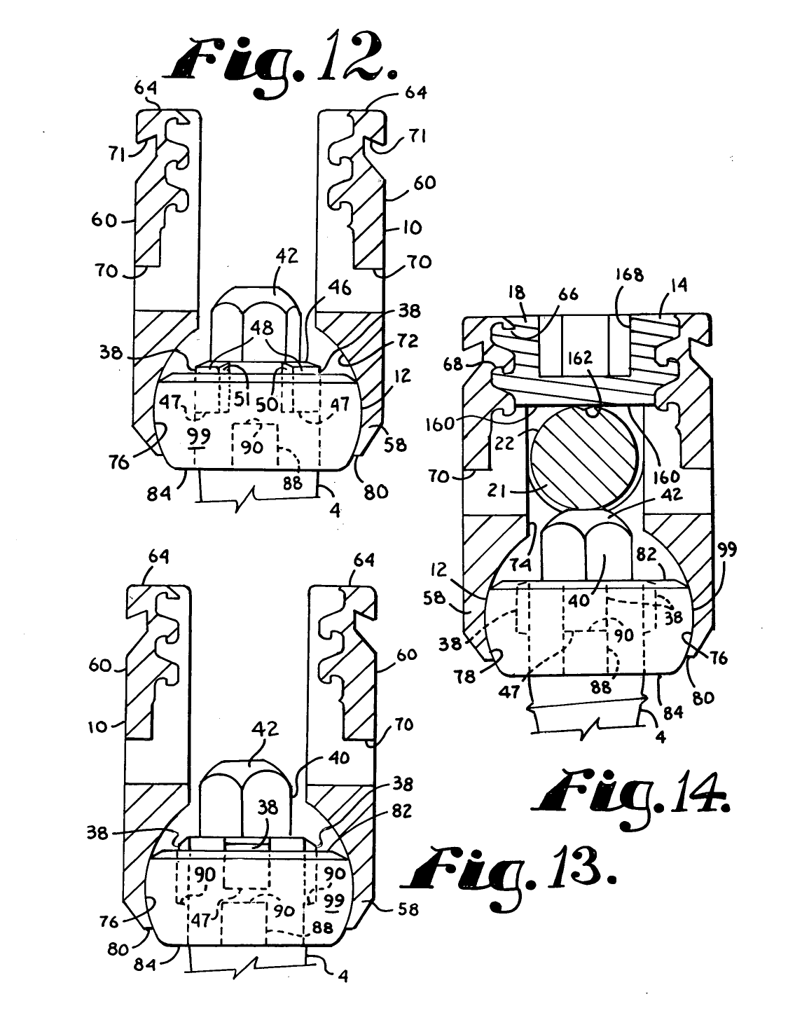

With particular reference to FIGS. 6-13, at the factory, the illustrated shank upper portion 8 is bottom loaded into the receiver 10 and the retainer 12 is top loaded into the receiver 10. Specifically, with reference to FIG. 6, the retainer 12 is initially loaded into the receiver 10 through the U-shaped channel 62 with the top surface 82 facing one of the arms 60 and the bottom surface 84 facing the opposite arm 60. With reference to FIG. 7, once the retainer 12 is disposed within the cavity 72, the retainer 12 is rotated to a shank-receiving position wherein the bottom surface 84 is facing the receiver lower opening 78. With reference to FIGS. 8-10 and 12, the shank upper portion 8 is aligned with respect to the retainer 12 such that the projections 38 are passed through the retainer 12 central bore 81 between each of the shelves 88, with each of the outer cylindrical surfaces 48 in slidable engagement with the inner cylindrical surface 86 of the retainer 12. With further reference to FIG. 12, once the bottom surfaces 47 of the projections 38 are disposed above and spaced from the seating surface 90 of the shelves 88, the shank 4 and/or the retainer 12 is rotated about the axis B until the projections 38 are disposed axially aligned and directly above the shelves 88 as shown in FIG. 13 with each of the bottom surfaces 47 of the projections 38 facing one of the seating surfaces 90 of the shelves 88. The shank upper portion 8 and the retainer 12 are moved along the axis B toward one another until the surfaces 47 engage the shelf surfaces 90 as illustrated in FIG. 11 and in phantom in FIG. 14. As is shown in FIG. 11, end portions of the shelf surfaces 90 extend beyond each of the projections 38 on either side thereof. At such locations, a tool may be used to press or crimp the retainer and/or shelf portions or a spot weld may be made to securely fix the retainer 12 to the shank upper portion 8 at the shelf surfaces 90. Adhesives, deformations and/or other crimping apparatus and methods (as, for example, illustrated and discussed below with respect to the bone screw assembly 201) may be used to ensure fixed engagement between the retainer 12 and the shank upper portion 8. At this time, the retainer 12 outer curved surface 99 is in sliding, pivotal engagement with the inner surface 76 of the receiver 10, allowing for a wide range of pivotal movement between the shank 4 and the receiver 10. Thus, both the shank 4 and the retainer 12 are in rotatable and swivelable engagement with the receiver 10, while the shank upper portion 8 and the lower aperture or neck 78 of the receiver 10 cooperate to maintain the shank body 6 in swivelable relation with the receiver 10. Only the retainer 12 is in slidable engagement with the receiver spherical seating surface 76. The shank upper end surface 42 and the shank body 6 are in spaced relation with the receiver 10. The shank body 6 can be rotated through a substantial angular rotation relative to the receiver 10, both from side to side and from front to rear so as to substantially provide a universal or ball joint.

In use, the assembly 1 is typically screwed into a bone, such as a vertebra (not shown), by rotation of the shank 4 using a driving tool (not shown that operably drives and rotates the shank 4 by engagement thereof with the tool engagement structure 40 that is in the form of a hexagonally shaped extension head.

The vertebra (not shown) may be pre-drilled to minimize stressing the bone and have a guide wire (not shown) that is shaped for the cannula 44 inserted to provide a guide for the placement and angle of the shank 4 with respect to the vertebra. A further tap hole may be made using a tap with the guide wire as a guide. Then, the assembly 1 is threaded onto the guide wire utilizing the cannulation bore 44 by first threading the wire into the bottom opening at the shank tip 32 and then out of the top opening located at the surface 42. The shank 4 is then driven into the vertebra, using the wire as a placement guide.

The longitudinal connecting member 21 is eventually positioned within the receiver U-shaped channel 62, and the closure structure or top 14 is then inserted into and advanced between the arms 60 so as to bias or push against the longitudinal connecting member 21. The break-off head 20 of the closure structure 14 is twisted to a preselected torque, for example 90 to 120 inch pounds, to urge the longitudinal connecting member 21 downwardly. The shank top end surface 42 is rounded to approximately equally extend upwardly into the channel 62 approximately the same amount no matter what degree of rotation exists between the shank 4 and receiver 10 and the surface 42 is sized to extend upwardly into the U-shaped channel 62. Therefore, the surface 42 is engaged by the longitudinal connecting member 21 and pushed downwardly toward the base 58 of the receiver 10 when the closure structure fastener portion 18 biases downwardly toward and onto the longitudinal connecting member 21. The downward pressure on the shank 4 in turn urges the retainer structure 12 downward toward the receiver seating surface 76, with the retainer structure surface 99 in frictional engagement with the receiver seating surface 76. As the closure structure fastener 18 presses against the longitudinal connecting member 21, the longitudinal connecting member 21 presses against the shank. The retainer structure 12 that is now rigidly attached to the shank 4 is in turn urged downwardly and becomes frictionally and rigidly attached to the receiver 10, fixing the shank body 6 in a desired angular configuration with respect to the receiver 10 and the longitudinal connecting member 21.

If removal of the assembly 1 and associated longitudinal connecting member 21 and closure structure 18 is necessary, disassembly is accomplished by using a driving tool of an Allen wrench type (not shown) mating with the aperture 168 and turned counterclockwise to rotate the fastener 18 and reverse the advancement thereof in the receiver 10. Then, disassembly of the assembly 1 is accomplished in reverse order to the procedure described previously herein for assembly.

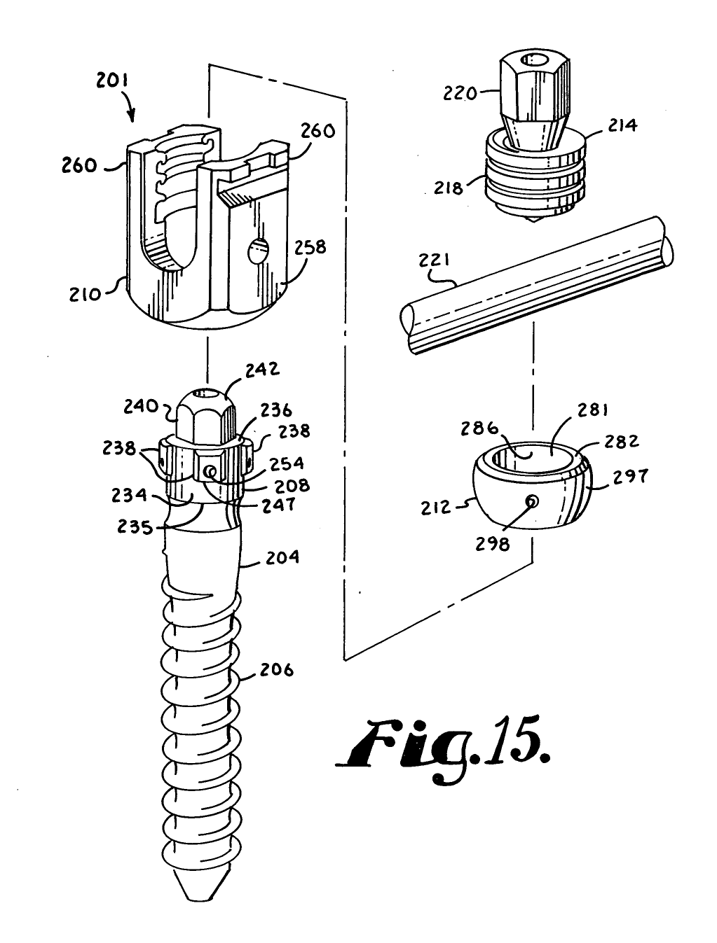

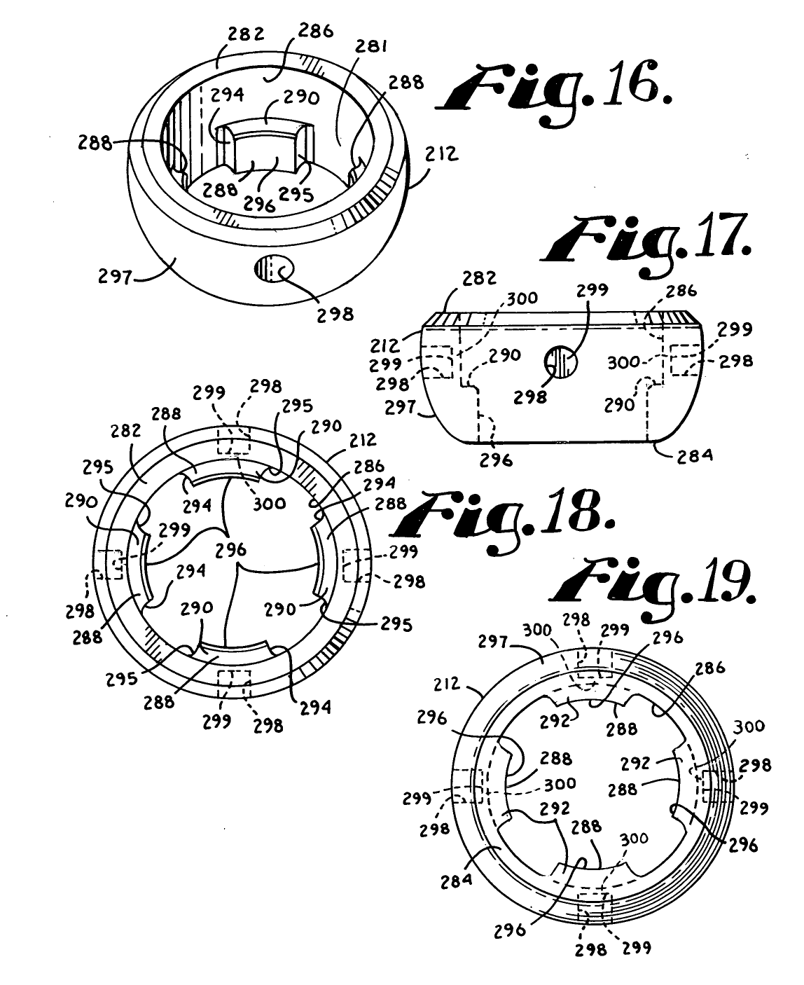

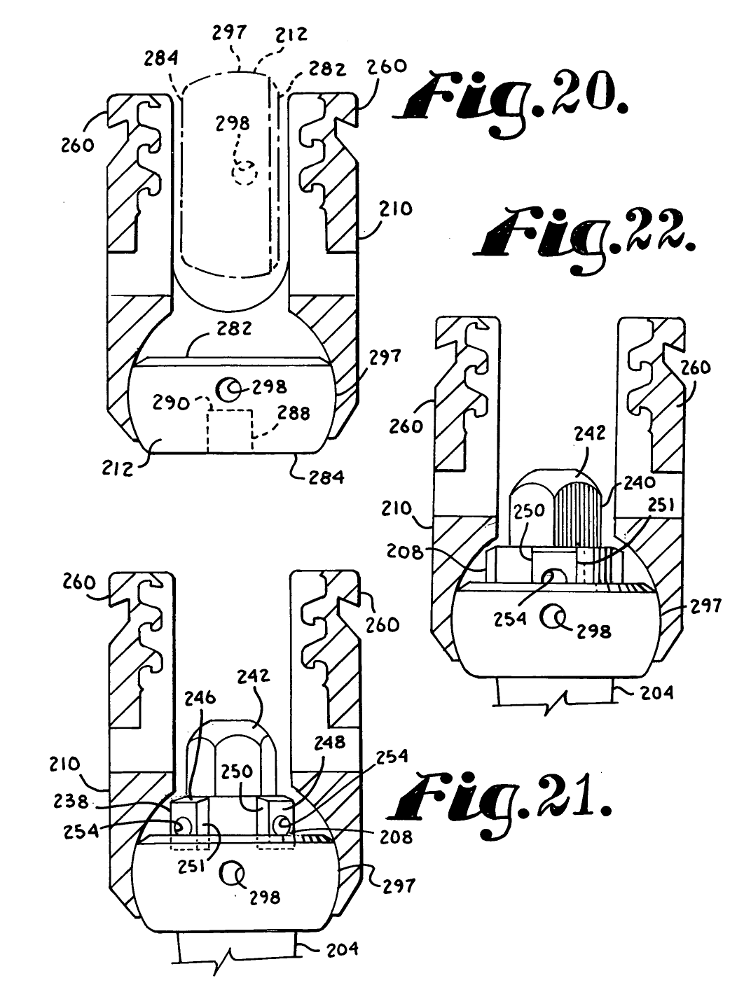

With reference to FIGS. 15-25, the reference number 201 generally designates an alternative polyaxial bone screw assembly according to the invention. The assembly 201 is substantially similar to the assembly 1 with the exception of additional structure for fixing the retainer structure to the upper portion of the bone screw shank. Thus, The assembly 201 includes a shank 204 that further includes a threaded body 206 integral with an upper portion 208; a receiver 210; a retainer structure 212 fixable to the upper portion 208 and swivelable with respect to the receiver 210; and a closure structure 214. The shank 204, receiver 210, and retainer structure 212 are typically factory assembled prior to implantation of the shank body 206 into a vertebra (not shown).

With further reference to FIG. 15, the closure structure 214 further includes a lower fastener portion 218 and a break-off head or top 220. The fastener portion 218 engages a longitudinal connecting member such as a rod 221, for example, having a cylindrical surface. The fastener portion 218 presses against the rod 221 that in turn presses upon the shank upper portion 208 which biases the retainer structure 212 into fixed frictional contact with the receiver 210, so as to fix the rod 221 relative to the vertebra (not shown). The receiver 210 and the shank 204 cooperate in such a manner that the receiver 210 and the shank 204 can be secured at any of a plurality of angles, articulations or rotational alignments relative to one another and within a selected range of angles both from side to side and from front to rear, to enable flexible or articulated engagement of the receiver 210 with respect to the shank 204 until both are locked or fixed relative to each other near the end of an implantation procedure.

With particular reference to FIGS. 15 and 21-25, the shank 204 is elongate, with the threaded body 206 and upper portion 208 having a cylindrical body 234 and a lower rim 235 being identical in form and function to the respective body 6 and upper portion 8 with cylindrical body 34 and rim 35 of the shank 4 previously described herein, with the exception that four shank projections 238 each have an outer cylindrical surface 248 having a centrally located aperture 254 formed therein for receiving crimped material from the retainer structure 212. The shank 204 otherwise includes an upper surface 236, a drive structure 240, a top domed surface 242, projection 238 top surfaces 246, bottom surfaces 247, and side surfaces 250, 251 that are the same or substantially similar to the upper surface 36, drive structure 40, top domed surface 42, projections top surfaces 46, bottom surfaces 47, and side surfaces 50 and 51 of the shank 4 of the assembly 1 previously described herein.

With particular reference to FIG. 15, the receiver 210 includes a base 258, arms 260 and all other features that are identical in form and function to the receiver 10 having the base 58 and arms 60 of the assembly 1.

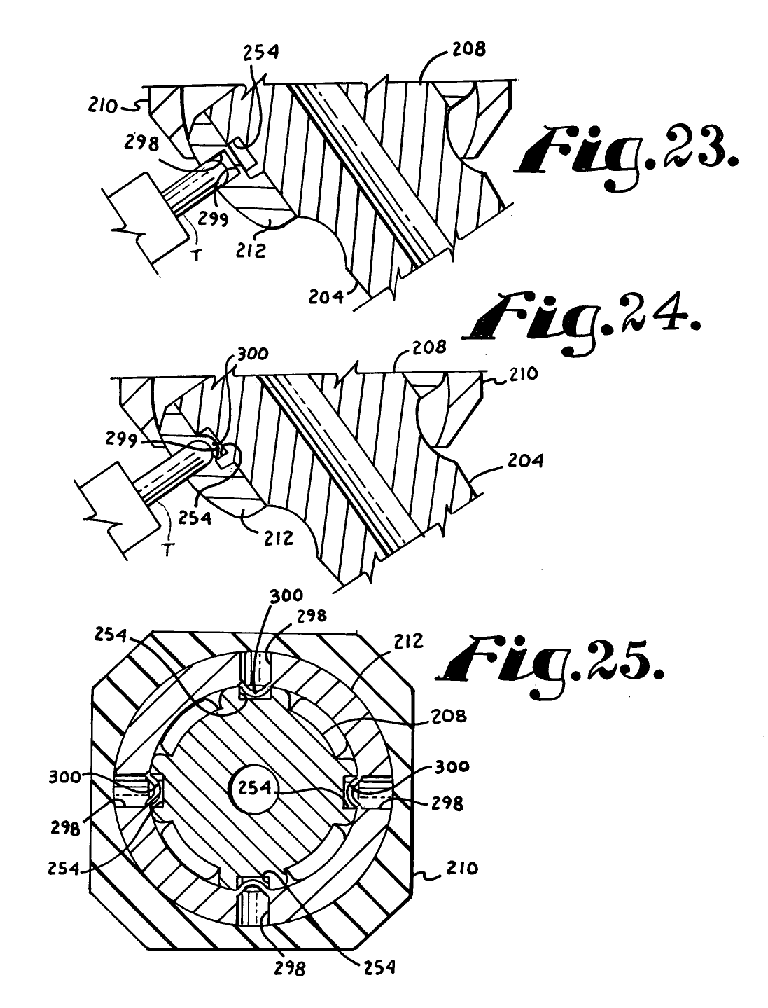

With particular reference to FIGS. 16-19, the retainer structure 212 includes a central bore 281, top surface 282, bottom surface 284, inner cylindrical surface 286, shelves 288, shelf seating surfaces 290, shelf bottom surfaces 292, shelf side surfaces 294, 295, shelf inner cylindrical surfaces 296, and retainer structure outer spherical surface 297 that are the same or similar in form and function to the respective retainer structure 12 central bore 81, top surface 82, bottom surface 84, inner cylindrical surface 86, shelves 88, shelf seating surfaces 90, shelf bottom surfaces 92, shelf side surfaces 94, 95, shelf inner cylindrical surfaces 96, and retainer structure outer spherical surface 99 previously described herein with respect to the assembly 1. Furthermore, formed in the retainer structure outer surface 297 are four apertures 298 that extend toward the inner surface 286, but do not extend therethrough. At a base 299 of each aperture 298, a thin section or wall 300 is deformable. The apertures 298 are located so that each wall 300 is alignable with one of the apertures 254 formed in the shank upper portion 208 as shown, for example, in FIGS. 23-25.

The illustrated closure top 214 is identical in form and function with the closure top 14 previously described herein with respect to the assembly 1. However, it is foreseen that a variety of closure tops, with or without break-off heads, may be used according to the invention that cooperate with the arms 260 of the receiver 210 to engage the rod 221 and press the rod 221 into engagement with the domed surface 242 of the shank upper portion 208.

The illustrated rod 221 is identical in form and function with the rod 21 previously described herein. However, a variety of longitudinal connecting members may be used with the bone screw 201, also as previously described with respect to the assembly 1.

With particular reference to FIGS. 20-25, at the factory, the illustrated shank upper portion 208 is bottom loaded into the receiver 210 and the retainer 212 is top loaded into the receiver 210 in the same manner as previously described herein with respect to the retainer 12 and the receiver 10 of the assembly 1. The shank upper portion 208 is aligned with respect to the retainer 212 such that the projections 238 are passed through the retainer 212 central bore 281 between each of the shelves 288, with each of the outer cylindrical surfaces 248 in slidable engagement with the inner cylindrical surface 286 of the retainer 212. Once the bottom surfaces 247 of the projections 238 are disposed above and spaced from the seating surface 290 of the shelves 288, the shank 204 and/or the retainer 212 is rotated about the receiver 210 axis until the projections 238 are disposed axially aligned and directly above the shelves 288 as shown in FIG. 22 with each of the bottom surfaces 247 of the projections 238 facing one of the seating surfaces 290 of the shelves 288. The shank upper portion 208 and the retainer 212 are moved along the receiver 210 axis toward one another until the surfaces 247 engage the shelf surfaces 290. As is shown in FIGS. 23 and 24, the shank upper portion 208 and retainer structure 212 are pivoted with respect to the receiver 210 to provide access to one of the apertures 298. A tool T is then inserted into the aperture 298 and presses or crimps the wall 300 into the aperture 254 of one of the projections 238. The shank 204 and attached retainer structure 212 are then rotated about the receiver 210 axis and pivoted to expose another of the apertures 298. With reference to FIG. 25, each of the apertures 298 are accessed in turn, to crimp or press each wall 300 into one of the apertures 254 located on the projections 238 of the shank upper portion 208 to securely fix the retainer structure 212 to the shank upper portion 208. At this time, the retainer 212 outer curved surface 297 is in sliding, pivotal engagement with the inner seating surface of the receiver 210, allowing for a wide range of pivotal movement between the shank 204 and the receiver 210. Thus, both the shank 204 and the retainer 212 are in rotatable and swivelable engagement with the receiver 210, while the shank upper portion 208 and the lower aperture or neck of the receiver 210 cooperate to maintain the shank body 206 in swivelable relation with the receiver 210. Only the retainer 212 is in slidable engagement with the receiver inner spherical seating surface. The shank upper end surface 242 and the shank body 206 are in spaced relation with the receiver 210. The shank body 206 can be rotated through a substantial angular rotation relative to the receiver 210, both from side to side and from front to rear so as to substantially provide a universal or ball joint. The assembly 201 may then be used in a manner described previously herein with respect to the assembly 1.

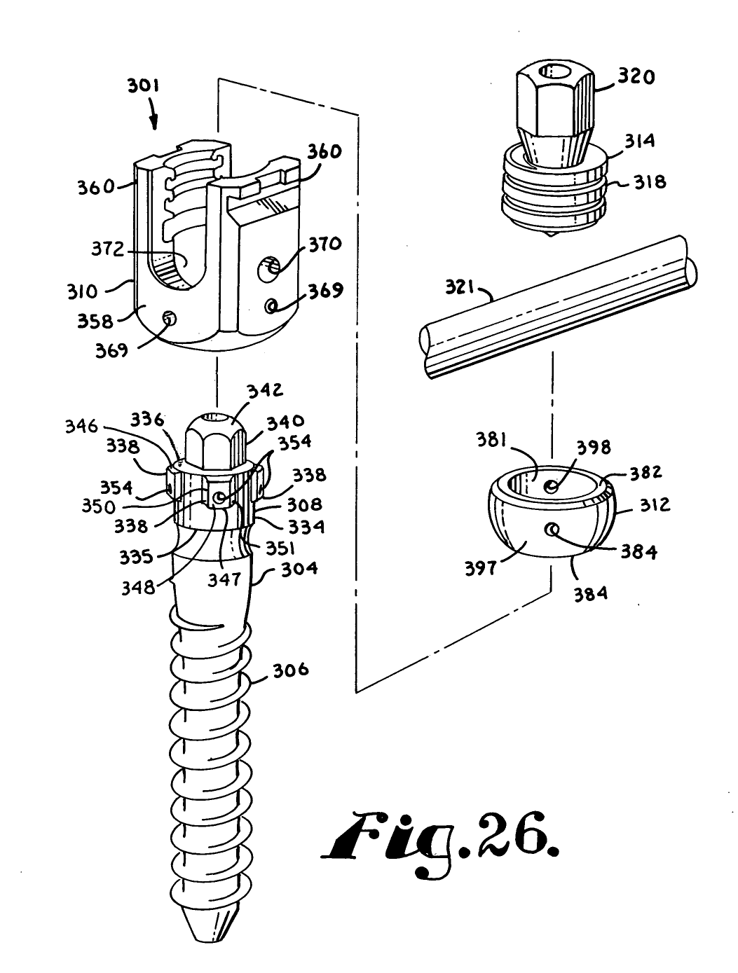

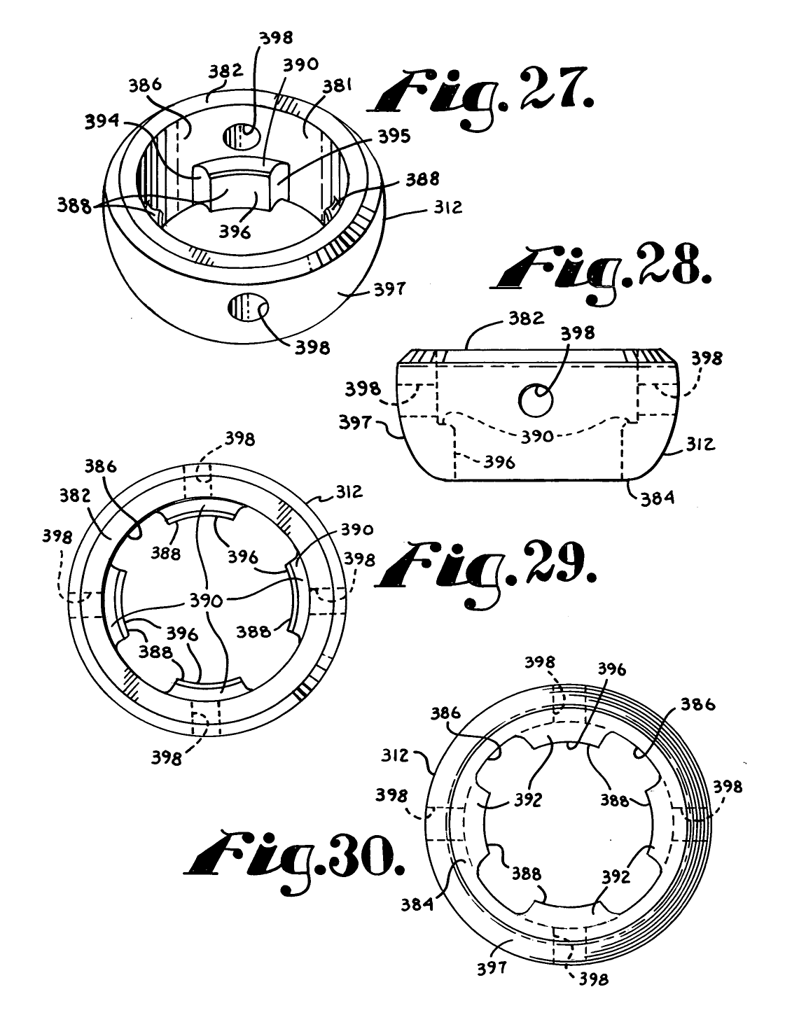

With reference to FIGS. 26-32, the reference number 301 generally designates another alternative polyaxial bone screw assembly according to the invention. The assembly 301 is substantially similar to the assembly 1 with the exception of additional structure for fixing the retainer structure to the upper portion of the bone screw shank. Thus, The assembly 301 includes a shank 304 that further includes a threaded body 306 integral with an upper portion 308; a receiver 310; a retainer structure 312 fixable to the upper portion 308 and swivelable with respect to the receiver 310; and a closure structure 314. The shank 304, receiver 310, and retainer structure 312 are typically factory assembled prior to implantation of the shank body 306 into a vertebra (not shown).

With further reference to FIG. 26, the closure structure 314 further includes a lower fastener portion 318 and a break-off head or top 320. The fastener portion 318 engages a longitudinal connecting member such as a rod 321, for example, having a cylindrical surface. The fastener portion 318 presses against the rod 321 that in turn presses upon the shank upper portion 308 which biases the retainer structure 312 into fixed frictional contact with the receiver 310, so as to fix the rod 321 relative to the vertebra (not shown). The receiver 310 and the shank 304 cooperate in such a manner that the receiver 310 and the shank 304 can be secured at any of a plurality of angles, articulations or rotational alignments relative to one another and within a selected range of angles both from side to side and from front to rear, to enable flexible or articulated engagement of the receiver 310 with respect to the shank 304 until both are locked or fixed relative to each other near the end of an implantation procedure.

With particular reference to FIGS. 26 and 31-32, the shank 304 is elongate, with the threaded body 306 and upper portion 308 having a cylindrical body 334 and a lower rim 335 being identical in form and function to the respective body 6 and upper portion 8 with cylindrical body 34 and rim 35 of the shank 4 previously described herein, with the exception that four shank projections 338 each have an outer cylindrical surface 348 having a centrally located aperture 354 formed therein for receiving a pin 399 that also extends through the retainer structure 312. The shank 304 otherwise includes an upper surface 336, a drive structure 340, a top domed surface 342, projection 338 top surfaces 346, bottom surfaces 347, and side surfaces 350, 351 that are the same or substantially similar to the upper surface 36, drive structure 40, top domed surface 42, projections top surfaces 46, bottom surfaces 47, and side surfaces 50 and 51 of the shank 4 of the assembly 1 previously described herein.

With particular reference to FIG. 26, the receiver 310 includes a base 358, two arms 360, tool receiving apertures 370, a cavity 372 and all other features that are identical in form and function to the receiver 10 having the respective base 58, arms 60, tool receiving apertures 70 and cavity 72 of the assembly 1. The receiver 310 further includes four through bores 369. Each through bore 369 is formed in the base 369 of the receiver 310 and extends in a radial direction to the cavity 372, each bore 369 communicating with both an outer surface of the base 358 and the receiver inner cavity 372 and further positioned to be aligned with apertures in the retainer structure 312 as will be described in greater detail below. Two of the through bores 369 are located below respective tool receiving apertures 370.

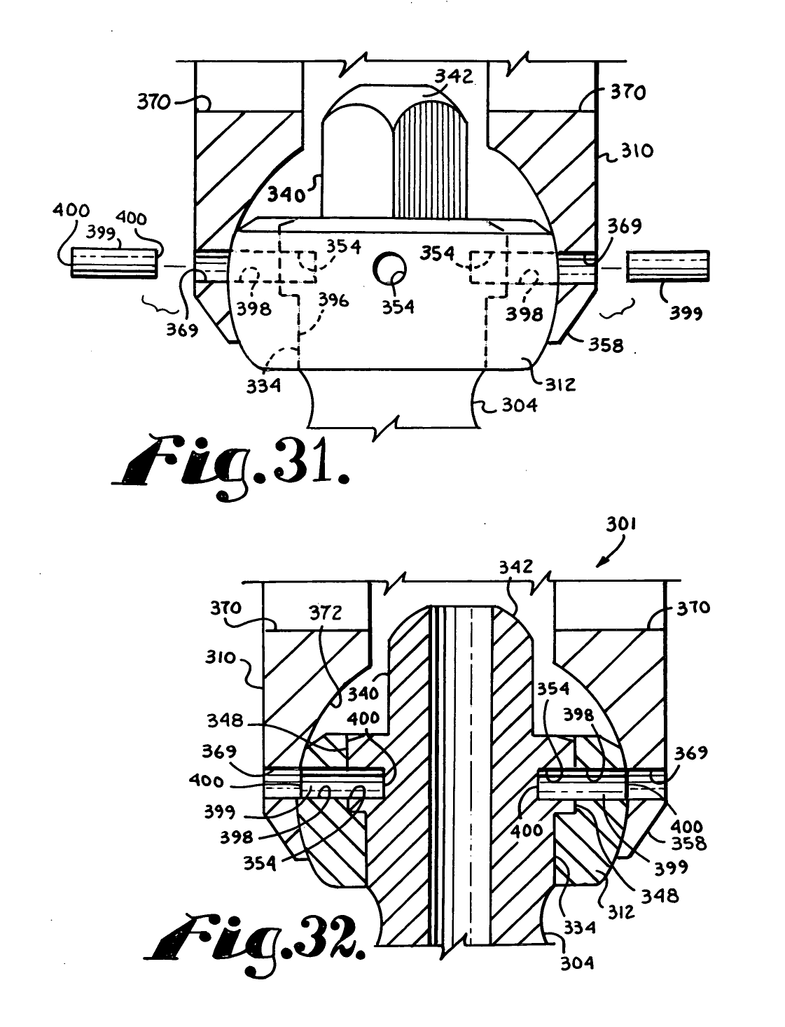

With particular reference to FIGS. 27-30, the retainer structure 312 includes a central bore 381, top surface 382, bottom surface 384, inner cylindrical surface 386, shelves 388, shelf seating surfaces 390, shelf bottom surfaces 392, shelf side surfaces 394, 395, shelf inner cylindrical surfaces 396, and retainer structure outer spherical surface 397 that are the same or similar in form and function to the respective retainer structure 12 central bore 81, top surface 82, bottom surface 84, inner cylindrical surface 86, shelves 88, shelf seating surfaces 90, shelf bottom surfaces 92, shelf side surfaces 94, 95, shelf inner cylindrical surfaces 96, and retainer structure outer spherical surface 99 previously described herein with respect to the assembly 1. Furthermore, formed in the retainer structure outer surface 397 are four apertures or through bores 398 that extend from the surface 397 completely through the inner surface 386. The apertures 398 are sized and shaped to closely receive and frictionally mate with the pin 399 having opposed ends 400. The apertures 398 are located so that each aperture 398 is alignable with one of the apertures 354 formed in the shank upper portion 308 as shown, for example, in FIGS. 31-32. The apertures 354 are also sized and shaped to closely receive and frictionally mate with one of the pins 300.

The illustrated closure top 314 is identical in form and function with the closure top 14 previously described herein with respect to the assembly 1. However, it is foreseen that a variety of closure tops, with or without break-off heads, may be used according to the invention that cooperate with the arms 360 of the receiver 310 to engage the rod 321 and press the rod 321 into engagement with the domed surface 342 of the shank upper portion 308.

The illustrated rod 321 is identical in form and function with the rod 21 previously described herein. However, a variety of longitudinal connecting members may be used with the bone screw 301, also as previously described with respect to the assembly 1.

With particular reference to FIGS. 26 and 31-32, at the factory, the illustrated shank upper portion 308 is bottom loaded into the receiver 310 and the retainer 312 is top loaded into the receiver 310 in the same manner as previously described herein with respect to the retainer 12 and the receiver 10 of the assembly 1. The shank upper portion 308 is aligned with respect to the retainer 312 such that the projections 338 are passed through the retainer 312 central bore 381 between each of the shelves 388, with each of the outer cylindrical surfaces 348 in slidable engagement with the inner cylindrical surface 386 of the retainer 312. Once the bottom surfaces 347 of the projections 338 are disposed above and spaced from the seating surface 390 of the shelves 388, the shank 304 and/or the retainer 312 is rotated about the receiver 310 axis until the projections 338 are disposed axially aligned and directly above the shelves 388 with each of the bottom surfaces 347 of the projections 338 facing one of the seating surfaces 390 of the shelves 388. The shank upper portion 308 and the retainer 312 are moved along the receiver 310 axis toward one another until the surfaces 347 engage the shelf surfaces 390. As is shown in FIGS. 31 and 32, the shank upper portion 308 and retainer structure 312 are then aligned within the receiver 310 to provide access to the apertures 298 through the apertures 369 of the receiver 310. One of the pins 399 is inserted into each of the apertures 369 and pressed into the retainer through one of the bores 384 and further into one of the shank apertures 354. Thus, a total of four pins 399 are press fit through receiver bores 369 and into aligned apertures 398 and 354 of the retainer and shank projection 338, respectively. The pins 399 each frictionally engage both the shank upper portion 308 and the retainer 312, providing a secure, fixed relationship between the shank upper portion 308 and the retainer 312. At this time, the retainer 312 outer curved surface 397 is in sliding, pivotal engagement with the inner seating surface of the receiver 310, allowing for a wide range of pivotal movement between the shank 304 and the receiver 310. Thus, both the shank 304 and the retainer 312 are in rotatable and swivelable engagement with the receiver 310, while the shank upper portion 308 and the lower aperture or neck of the receiver 310 cooperate to maintain the shank body 306 in swivelable relation with the receiver 310. Only the retainer 312 is in slidable engagement with the receiver inner spherical seating surface. The shank upper end surface 342 and the shank body 306 are in spaced relation with the receiver 310. The shank body 306 can be rotated through a substantial angular rotation relative to the receiver 310, both from side to side and from front to rear so as to substantially provide a universal or ball joint. The assembly 301 may then be used in a manner described previously herein with respect to the assembly 1.

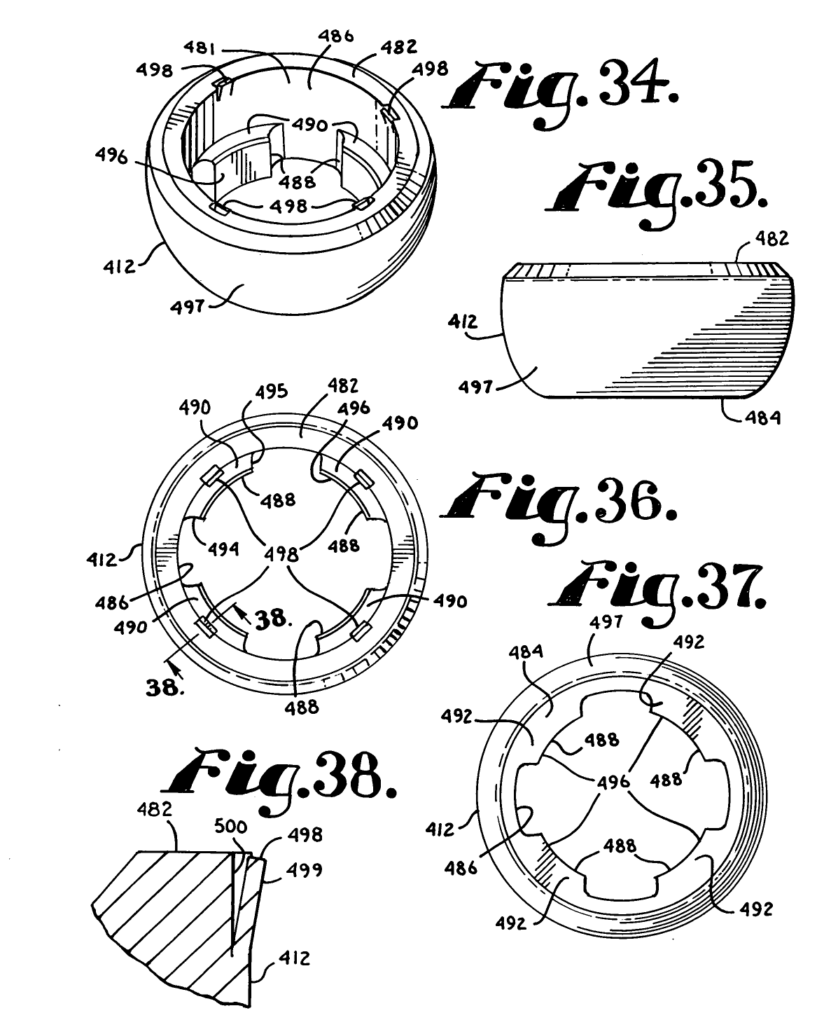

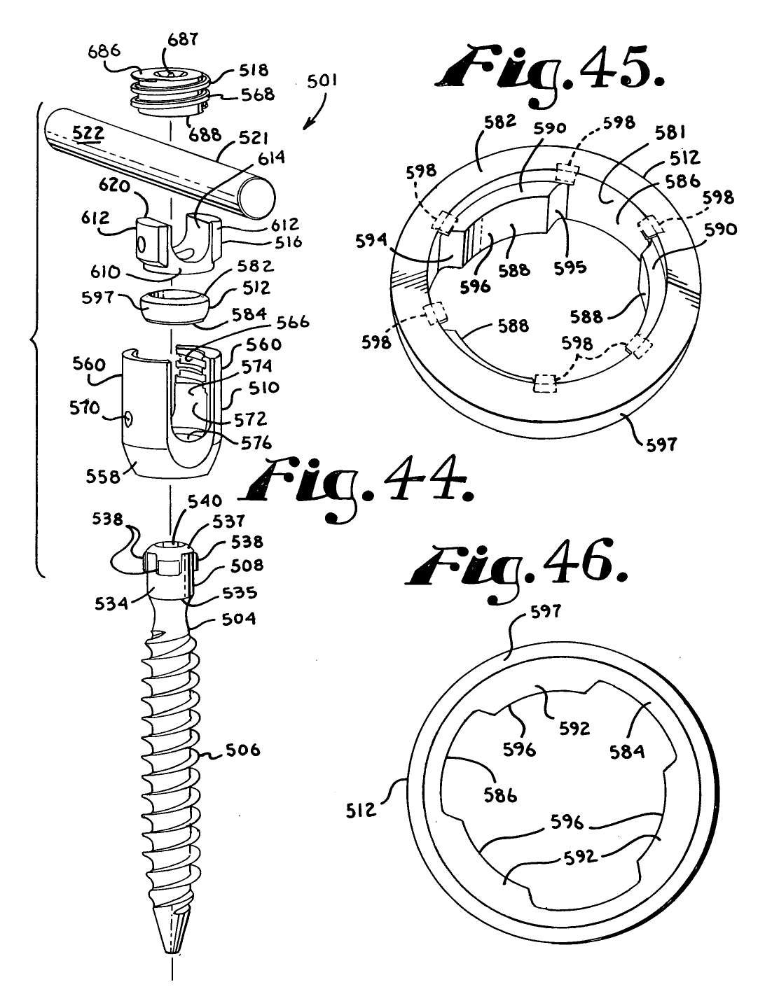

With reference to FIGS. 33-43, the reference number 401 generally designates another alternative polyaxial bone screw assembly according to the invention. The assembly 401 is substantially similar to the assembly 1 with the exception of additional structure for fixing the retainer structure to the upper portion of the bone screw shank. Thus, the assembly 401 includes a shank 404 that further includes a threaded body 406 integral with an upper portion 408; a receiver 410; a retainer structure 412 fixable to the upper portion 408 and swivelable with respect to the receiver 410; and a closure structure 414. The shank 404, receiver 410, and retainer structure 412 are typically factory assembled prior to implantation of the shank body 406 into a vertebra (not shown).

With further reference to FIG. 33, the closure structure 414 further includes a lower fastener portion 418 and a break-off head or top 420. The fastener portion 418 engages a longitudinal connecting member such as a rod 421, for example, having a cylindrical surface. The fastener portion 418 presses against the rod 421 that in turn presses upon the shank upper portion 408 which biases the retainer structure 412 into fixed frictional contact with the receiver 410, so as to fix the rod 421 relative to the vertebra (not shown). The receiver 410 and the shank 404 cooperate in such a manner that the receiver 410 and the shank 404 can be secured at any of a plurality of angles, articulations or rotational alignments relative to one another and within a selected range of angles both from side to side and from front to rear, to enable flexible or articulated engagement of the receiver 410 with respect to the shank 404 until both are locked or fixed relative to each other near the end of an implantation procedure.

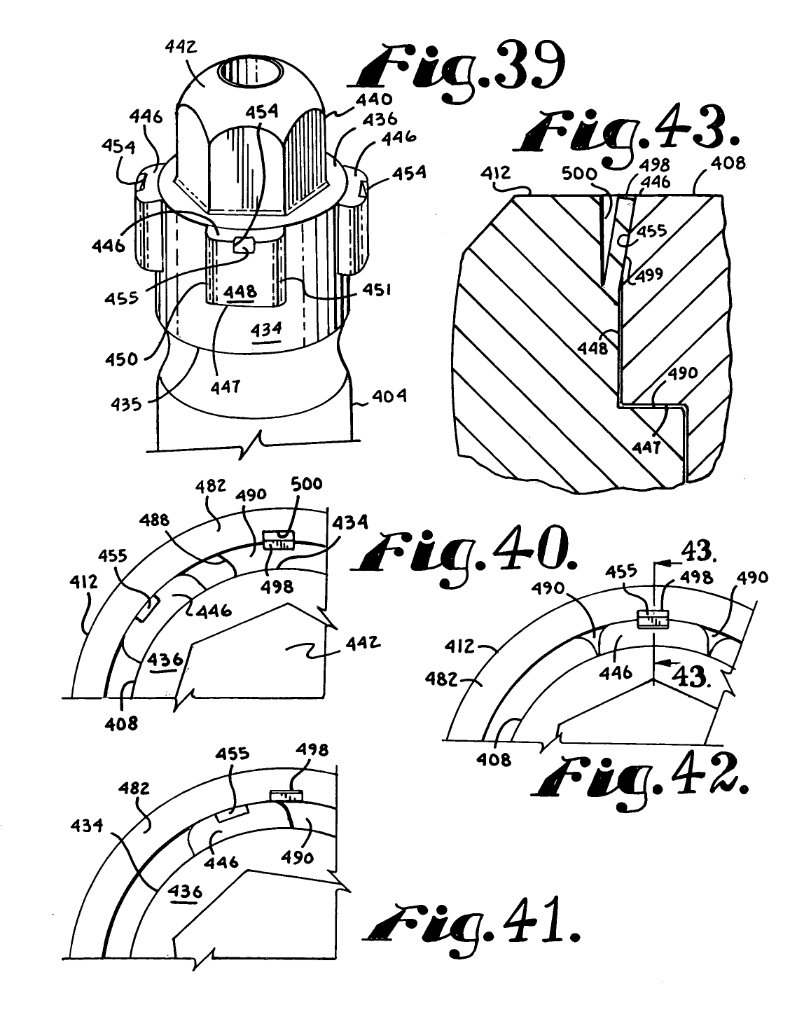

With particular reference to FIGS. 33 and 39, the shank 404 is elongate, with the threaded body 406 and upper portion 408 having a cylindrical body 434 and a lower rim 435 being identical in form and function to the respective body 6 and upper portion 8 with cylindrical body 34 and rim 35 of the shank 4 previously described herein, with the exception that four shank projections 438 each have an outer cylindrical surface 448 having an upper cut or capture notch 454 with a planar surface 455 formed at an upper edge thereof for frictionally engaging a deformable, but stiff crimp tab 498 of the retainer structure 412. The shank 404 otherwise includes an upper surface 436, a drive structure 440, a top domed surface 442, projection 438 top surfaces 446, bottom surfaces 447, and side surfaces 450, 451 that are the same or substantially similar to the upper surface 36, drive structure 40, top domed surface 42, projections top surfaces 46, bottom surfaces 47, and side surfaces 50 and 51 of the shank 4 of the assembly 1 previously described herein. With particular reference to FIG. 39, the notch 454 is centrally located at the juncture of each projection top surface 446 and outer cylindrical surface 448.

With particular reference to FIG. 33, the receiver 410 includes a base 458 and two arms 460 and all other features that are identical in form and function to the receiver 10 having the respective base 58, arms 60 and all other features of the receiver 10 previously described herein with respect to the assembly 1.

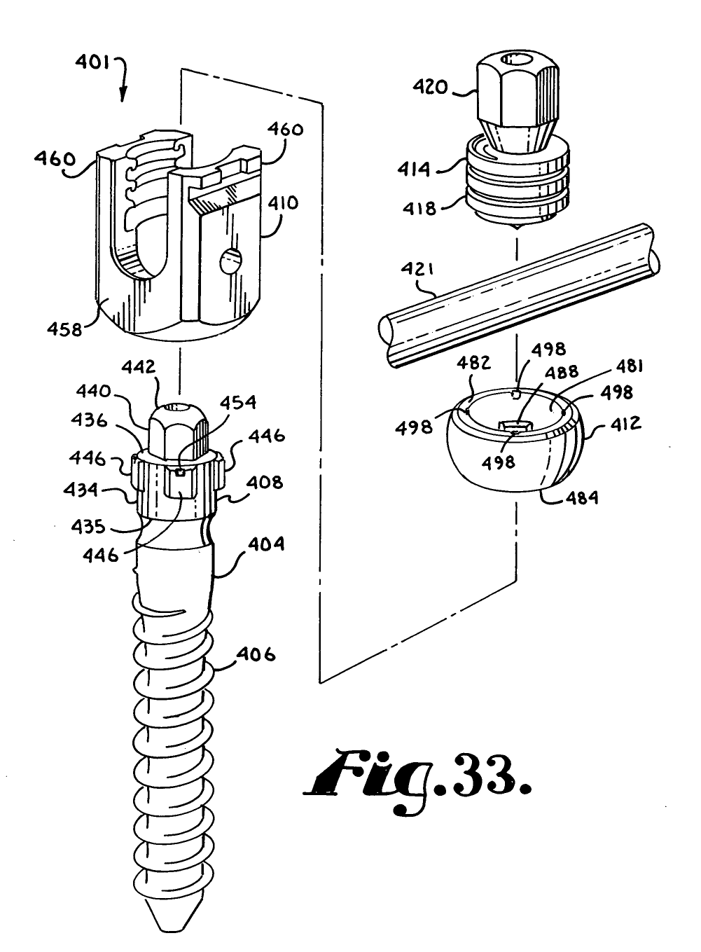

With particular reference to FIGS. 34-38, the retainer structure 412 includes a central bore 481, top surface 482, bottom surface 484, inner cylindrical surface 486, shelves 488, shelf seating surfaces 490, shelf bottom surfaces 492, shelf side surfaces 494, 495, shelf inner cylindrical surfaces 496, and retainer structure outer spherical surface 497 that are the same or similar in form and function to the respective retainer structure 12 central bore 81, top surface 82, bottom surface 84, inner cylindrical surface 86, shelves 88, shelf seating surfaces 90, shelf bottom surfaces 92, shelf side surfaces 94, 95, shelf inner cylindrical surfaces 96, and retainer structure outer spherical surface 99 previously described herein with respect to the assembly 1. Furthermore, a crimp-tab 498 is cut or otherwise formed in the top surface 482 and extends inwardly toward the bore 481 at the inner cylindrical surface 486. Each crimp-tab 498 is located centrally above each shelf 488 and includes a mating surface 499 for engaging the notch surface 455 of the shank upper portion 408. Each crimp-tab 498 forms a space 500, after the deformable tab 498 is bent outwardly radially into notch 455 during assembly. The tab 498 can also be pre-bent into the position shown in FIG. 38. When the shank is rotated into position, the tab 498 can move back into space 500 and then snap into the notch 455 to stabilize the retainer.

The illustrated closure top 414 is identical in form and function with the closure top 14 previously described herein with respect to the assembly 1. However, it is foreseen that a variety of closure tops, with or without break-off heads, may be used according to the invention that cooperate with the arms 460 of the receiver 410 to engage the rod 421 and press the rod 421 into engagement with the domed surface 442 of the shank upper portion 408.

The illustrated rod 421 is identical in form and function with the rod 21 previously described herein. However, a variety of longitudinal connecting members may be used with the bone screw 401, also as previously described with respect to the assembly 1.

With particular reference to FIGS. 33 and 40-43, at the factory, the illustrated shank upper portion 408 is bottom loaded into the receiver 410 and the retainer 412 is top loaded into the receiver 410 in the same manner as previously described herein with respect to the retainer 12 and the receiver 10 of the assembly 1. The shank upper portion 408 is aligned with respect to the retainer 412 such that the projections 438 are passed through the retainer 412 central bore 481 between each of the shelves 488, with each of the outer cylindrical surfaces 448 in slidable engagement with the inner cylindrical surface 486 of the retainer 412. Once the bottom surfaces 447 of the projections 438 are level with or disposed somewhat above and spaced from the seating surface 490 of the shelves 488, the shank 404 and/or the retainer 412 is rotated about the receiver 410 axis until the tabs 498 are aligned with and snapped into the notches 454 or are bent down into the notches 454. The shank upper portion 408 and the retainer 412 are then moved toward one another, if needed, along the receiver 410 axis toward one another until the surfaces 447 fully frictionally engage the shelf surfaces 490. At this time, the retainer 412 outer curved surface 497 is in sliding, pivotal engagement with the inner seating surface of the receiver 410, allowing for a wide range of pivotal movement between the shank 404 and the receiver 410. Thus, both the shank 404 and the retainer 412 are in rotatable and swivelable engagement with the receiver 410, while the shank upper portion 408 and the lower aperture or neck of the receiver 410 cooperate to maintain the shank body 406 in swivelable relation with the receiver 410. Only the retainer 412 is in slidable engagement with the receiver inner spherical seating surface. The shank upper end surface 442 and the shank body 406 are in spaced relation with the receiver 410. The shank body 406 can be angulated or pivoted through a substantial angular motion relative to the receiver 410, both from side to side and from front to rear so as to substantially provide a universal or ball joint. The assembly 401 may then be used in a manner described previously herein with respect to the assembly 1.

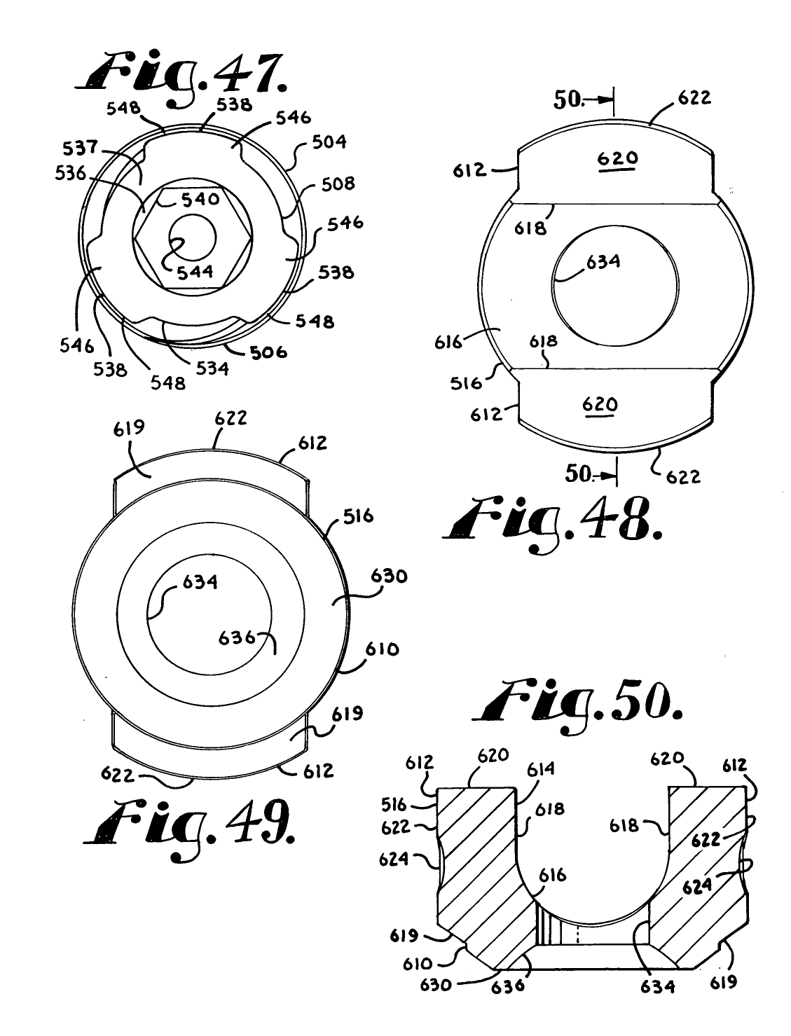

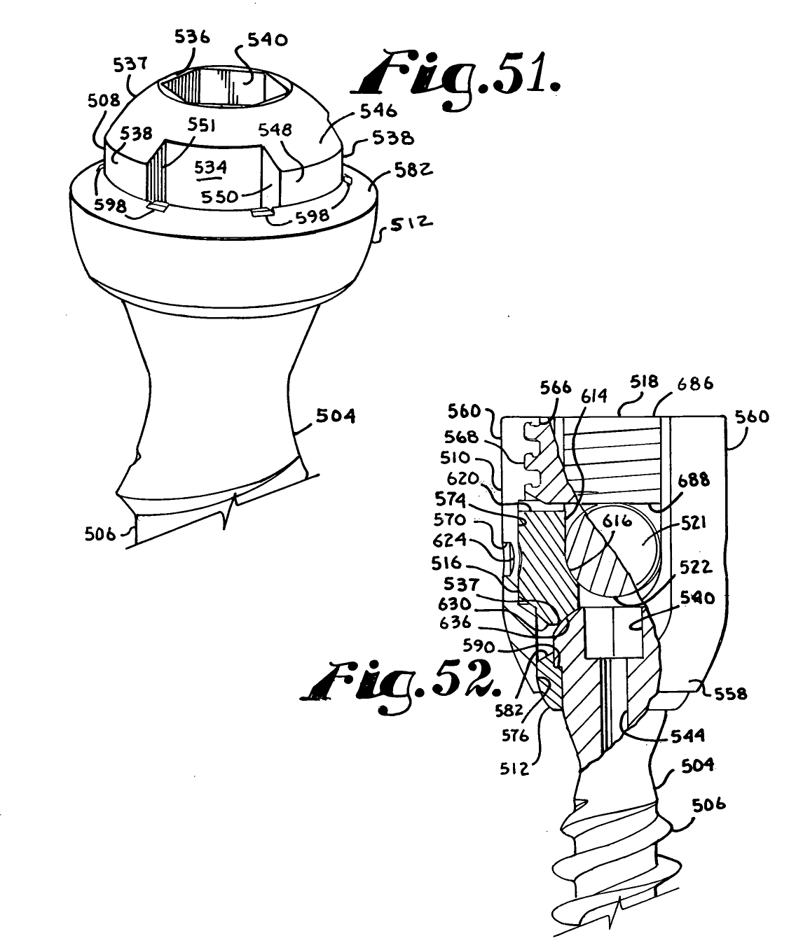

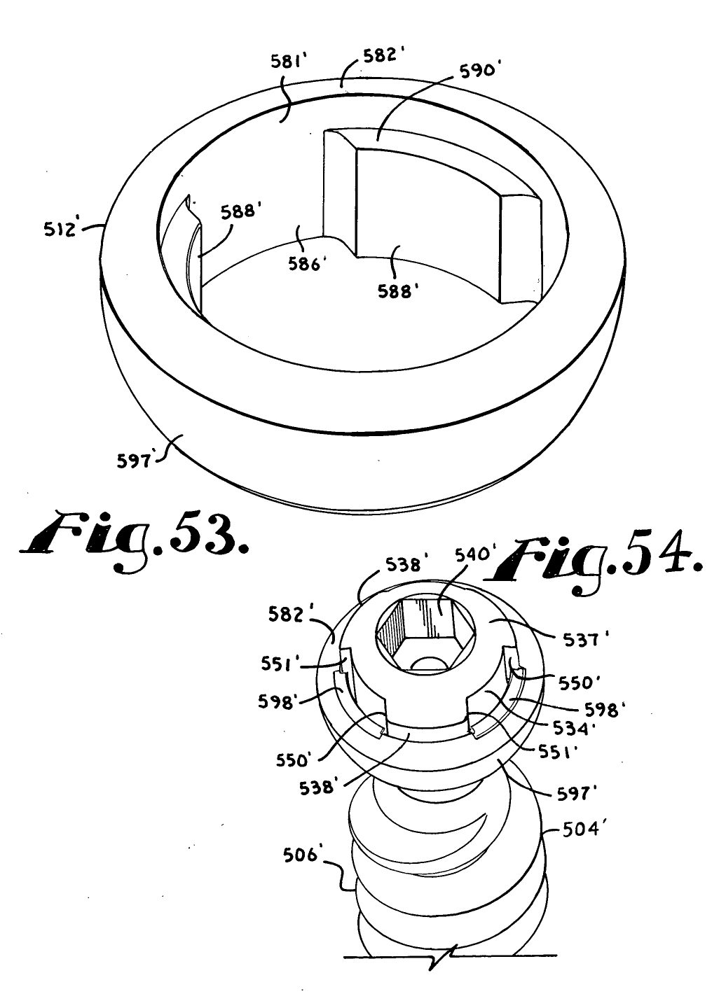

With reference to FIGS. 44-52, the reference number 501 generally represents another alternative embodiment of a polyaxial bone screw apparatus or assembly according to the present invention. The assembly 501 includes a shank 504 that further includes a threaded body 506 integral with an upper portion 508; a receiver 510; a retainer structure 512 fixable to the upper portion 508 and swivelable with respect to the receiver 510; a lower pressure insert 516; and a one-piece closure structure 514. The shank 504, receiver 510, retainer structure 512 and pressure insert 516 are typically factory assembled prior to implantation of the shank body 506 into a vertebra (not shown).

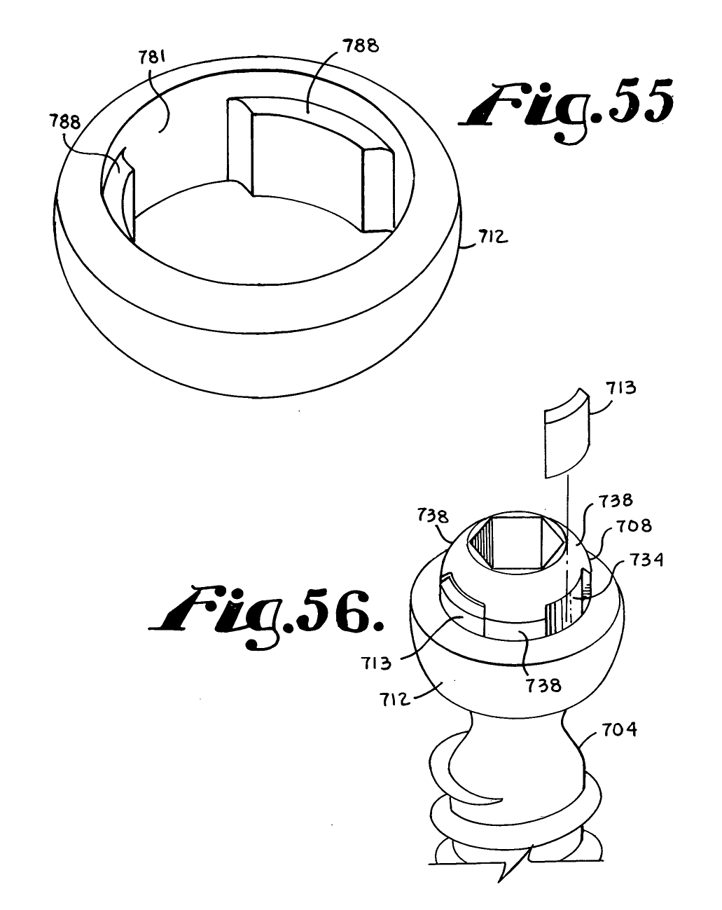

With further reference to FIG. 44, the closure structure 518 engages a longitudinal connecting member such as a rod 521, for example, having a cylindrical surface 522 shown in FIGS. 44 and 52. The closure 518 presses against the rod 521 that in turn presses upon the pressure insert 516 that in turn presses upon the shank upper portion 508 which biases the retainer structure 512 into fixed frictional contact with the receiver 510, so as to fix the rod 521 relative to the vertebra (not shown). It is foreseen that in some embodiments of the invention, especially when using a deformable rod, the closure top and the lower pressure insert may be configured such that the bottom of the closure top directly engages an upper end surface of the lower pressure insert that in turn presses upon the shank upper portion to bias the retainer structure into fixed frictional contact with the receiver. The receiver 510 and the shank 504 cooperate in such a manner that the receiver 510 and the shank 504 can be secured at any of a plurality of angles, articulations or rotational alignments relative to one another and within a selected range of angles both from side to side and from front to rear, to enable flexible or articulated engagement of the receiver 510 with respect to the shank 4 until both are locked or fixed relative to each other near the end of an implantation procedure.

With particular reference to FIGS. 44, 47 and 51-52, the shank 504 is elongate and is substantially similar to the shank 4 previously described herein with respect to the assembly 1, with the threaded body 506 being substantially similar to the threaded body 6 of the assembly 1. Furthermore, the upper portion 508 includes a cylindrical body 534 and a lower rim 535, and projections 538 that are substantially similar in form and function to the cylindrical body 34, rim 35 and projections 38 of the assembly 1. However, the upper portion 508 includes only three equally spaced projections 538. The projections 538 are adjacent to an upper domed surface 537 that terminates at an upper to flat annular surface 536. Formed in the annular surface 536 is an internal drive feature 540. A cannulation bore 544 communicates with the drive feature 540 and extends along and through an entire length of the shank body 506. The upper domed surface 537 has the same radius as each projection top surfaces 546, resulting in a substantially domed engagement surface that is sized and shaped to frictionally engage and mate with a bottom spherical surface 636 of the pressure insert 516 as will be described in greater detail below. Each projection 538 further includes a bottom surface 547, an outer cylindrical surface 548 and opposed side surfaces 550 and 551 that are substantially similar in form and function with the bottom surface 47, cylindrical surface 48 and respective side surfaces 50 and 51 of the projections 38 of the upper portion 8 of the assembly 1 previously described herein. While not required in accordance with the practice of the invention, the surfaces 537 and 546 may be scored or knurled to further increase frictional positive mating engagement between the shank upper portion 508 and the lower pressure insert 516. The cannulation bore 544 provides a passage through the shank 504 interior for a length of wire (not shown) inserted into the vertebra (not shown) prior to the insertion of the shank body 506, the wire providing a guide for insertion of the shank body 506 into the vertebra (not shown).

Referring to FIGS. 44 and 52, the receiver 510 is substantially similar in form and function to the receiver 10 previously described in detail herein. Thus, the receiver 510 includes features such as a receiver base 558, arms 560, inner guide and advancement structure 566, inner cavity 572 that includes a spherical seat 576 that are identical or substantially similar to the respective base 58, arms 60, guide and advancement structure 66, inner cavity 572 and spherical seat 76 of the receiver 10 of the assembly 1, as previously described herein. It is noted that the receiver 510 is sized and shaped slightly differently from the receiver 10 in order to receive and hold the pressure insert 516 as will be described in greater detail below. Furthermore, the receiver includes partial apertures 570 formed in the arms 560 that are sized, shaped and positioned to cooperate with apertures located on the pressure insert 516. A thin wall of material from the receiver 510 may be pressed or crimped into the apertures of the pressure insert 516 to hold the insert 516 in alignment within the receiver 510. The guide and advancement structure 566 is sized and shaped to mate with a helical guide and advancement structure 568 located on the closure top 518. Tool mating structure can also be welded to the receiver 510.

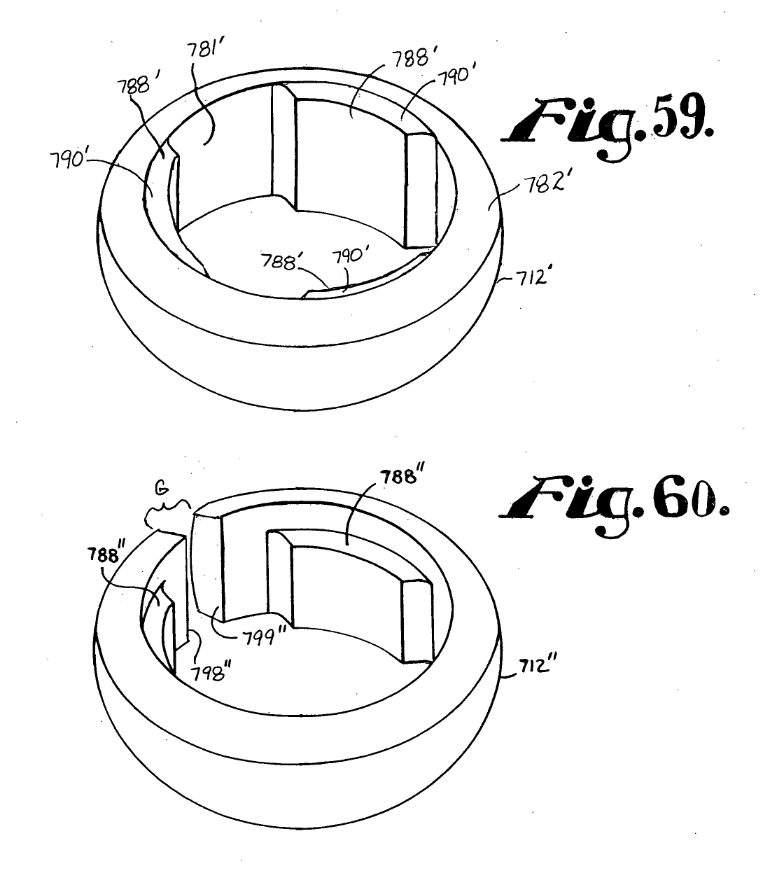

With particular reference to FIGS. 44-46, the retainer structure 512 includes a central bore 581, top surface 582, bottom surface 584, inner cylindrical surface 586, shelves 588, shelf seating surfaces 590, shelf bottom surfaces 592, shelf side surfaces 594, 595, shelf inner cylindrical surfaces 596, and retainer structure outer spherical surface 597 that are the same or substantially similar in form and function to the respective retainer structure 12 central bore 81, top surface 82, bottom surface 84, inner cylindrical surface 86, shelves 88, shelf seating surfaces 90, shelf bottom surfaces 92, shelf side surfaces 94, 95, shelf inner cylindrical surfaces 96, and retainer structure outer spherical surface 99 previously described herein with respect to the assembly 1. It is noted that the retainer top surface 582 is sloped to better provide clearance between the retainer 512 and the lower pressure insert 516 as shown in FIG. 52 and also illustrated in FIG. 58. Furthermore, cut-and-crimp portions 598 (shown in phantom) are eventually made in the top surface 582 and along the inner surface 586 after the retainer 512 is assembled with the shank upper portion 508 as will be described in greater detail below.

With particular reference to FIGS. 44 and 48-50, the lower compression or pressure insert 516 includes a substantially cylindrical base body 610 integral with a pair of upstanding arms 612. The body 610 and arms 612 form a generally U-shaped, open, through-channel 614 having a lower seat 616 sized and shaped to closely, snugly engage the rod 521. It is foreseen that an alternative embodiment may be configured to include planar holding surfaces that closely hold a square or rectangular bar as well as hold a cylindrical rod-shaped or corded longitudinal connecting member. The arms 612 disposed on either side of the channel 614 extend outwardly from the body 610. The arms 612 are sized and configured for placement near a run-out below the guide and advancement structure 566 at the receiver inner arms. In some embodiments of the invention, the arms 612 may be extended and the closure top configured such the arms 612 ultimately directly engage the closure top for locking of the polyaxial mechanism. In the present embodiment, the arms 612 include top surfaces 620 that are ultimately positioned in spaced relation with the closure top 518 so that the closure top 518 frictionally engages and holds the rod 521, pressing the rod 521 downwardly against the seating surface 616, the insert 516 in turn pressing against the domed top 537 of the shank 504 to lock the polyaxial mechanism of the bone screw assembly 501. Each arm 612 further includes inner planar walls 618, sloping lower surfaces 619 and partially cylindrical outer surface portions 622 sized and shaped to fit within the receiver 510 cavity 572 at a location below the guide and advancement structures 566. The cylindrical surfaces 622 are disposed substantially perpendicular to the respective adjacent top surfaces 620. Each of the outer surfaces 622 further includes a recess or partial aperture 624 sized and shaped to receive holding tabs or, in the illustrated embodiment, crimped material from the receiver 510 at the partial apertures 570. In other embodiments of the invention, the receiver 510 may be equipped with spring tabs that snap into the recesses 624 to hold the insert 516 in place with respect to rotation. The recesses 624 are preferably oval or elongate such that some desirable upward and downward movement of the insert 516 with respect to a central axis of the receiver 510 is not prohibited.

The compression insert 516 further includes an inner cylindrical surface 634 that forms a through bore sized and shaped to receive a driving tool (not shown) therethrough that engages the shank internal drive feature 540 when the shank body 506 is driven into bone. The inner surface 634 runs between the seating surface 616 and an inner curved, annular, radiused or semi-spherical surface 636. The surface 636 is sized and shaped to slidingly and pivotally mate with and ultimately fix against the annular domed surface 537 and adjacent projection top surfaces 546 of the shank upper portion 508. Thus, a radius of the surface 636 is the same or substantially similar to the radius of the surface 537 and the three projection top surfaces 546. The surface 636 may include a roughening or surface finish to aid in frictional contact between the surface 636 and the surfaces 537 and 546, once a desired angle of articulation of the shank 506 with respect to the receiver 510 is reached. Adjacent to the inner surface 536 is a bottom rim or edge 630. The surface portion or portions 619 run between the bottom rim 630 and the base body 610 and/or the arm outer surfaces 622. These surfaces include v-shaped cuts or other contours to provide clearance between the insert 516 and the retainer 514 during assembly and articulation of the bone screw shank 504 with respect to the receiver 510.

The pressure insert body 610 located between the arms 612 has an outer diameter slightly smaller than a diameter between crests of the guide and advancement structure 566 of the receiver 510 allowing for top loading of the compression insert 516 into the receiver 510 cavity 572, with the arms 612 of the insert 516 being located between the receiver arms 560 during insertion of the insert 516 into the receiver 510. Once located between the guide and advancement structure 566 above and the shank upper portion 508 below, the insert 516 is rotated into place about the receiver axis until the arms 612 are directly below the guide and advancement structure 566. After the insert 516 is rotated into such position, a tool (not shown) may be inserted into the receiver apertures 570 to press the thin receiver walls into the insert recesses 624. The lower compression insert 516 is sized such that the insert 516 is ultimately received within a substantially cylindrical surface portion 574 of the receiver 510 below the guide and advancement structure 566. The receiver 510 fully receives the lower compression insert 516 and supports and blocks the arms 612 of the structure 516 from spreading or splaying in any direction. It is noted that assembly of the shank 504 with the retainer 512 within the receiver 510, followed by insertion of the lower compression insert 516 into the receiver 510 are assembly steps typically performed at the factory, advantageously providing a surgeon with a polyaxial bone screw with the lower insert 516 already held in alignment with the receiver 510 and thus ready for insertion into a vertebra.

The compression or pressure insert 516 ultimately seats exclusively on the surface 537 of the shank upper portion 508. The assembly may be configured so that the insert 516 extends at least partially into the receiver U-shaped channel such that the seating surface 616 substantially contacts and engages an adjacent surface 522 of the rod 521 when the rod 521 is placed in the receiver 510 and the closure structure or top 518 is tightened against the rod, the illustrated rod 521 being fixedly held in spaced relation with, but not engaging a lower surface of the U-shaped channel of the receiver 510.

With particular reference to FIGS. 10-12, the elongate connecting member illustrated in the drawing figures is the hard, solid cylindrical rod 521 of circular cross-section. However, longitudinal connecting members for use with the assembly 501 may take a variety of shapes, including but not limited to rods or bars of oval, rectangular or other curved or polygonal cross-section. The shape of receiving surfaces defining the insert 516 channel may be modified so as to closely hold, and if desired, fix the longitudinal connecting member to the assembly 501. Furthermore, the longitudinal connector 521 may be a component of a longer overall dynamic stabilization connecting member, with cylindrical or bar-shaped portions sized and shaped for being received by the compression insert 516 of the receiver 510 having a u-shaped channel (or rectangular- or other-shaped channel) for closely receiving the longitudinal connecting member. The longitudinal connecting member 521 may be integral or otherwise fixed to a bendable or damping component that is sized and shaped to be located between adjacent pairs of bone screw assemblies 501, for example. A damping component or bumper may be attached to the member 521 at one or both sides of the bone screw assembly 501. A rod or bar (or rod or bar component) of a longitudinal connecting member may be made of a variety of materials ranging from deformable plastics to hard metals, depending upon the desired application. Thus, bars and rods of the invention may be made of materials including, but not limited to metal and metal alloys including but not limited to stainless steel, titanium, titanium alloys and cobalt chrome; or other suitable materials, including plastic polymers such as polyetheretherketone (PEEK), ultra-high-molecular weight-polyethylene (UHMWP), polyurethanes and composites, including composites containing carbon fiber, natural or synthetic elastomers such as polyisoprene (natural rubber), and synthetic polymers, copolymers, and thermoplastic elastomers, for example, polyurethane elastomers such as polycarbonate-urethane elastomers.

With reference to FIGS. 44 and 52, the closure structure or closure top 518 can be any of a variety of different types of closure structures for use in conjunction with the present invention with suitable mating structure on the receiver upstanding arms 560. In the embodiment shown, the closure top 518 is rotatably received between the spaced arms 560 of the receiver 510. The illustrated closure structure 518 is substantially cylindrical and includes the previously mentioned outer helically wound guide and advancement structure 568 in the form of a flange form that operably joins with the guide and advancement structure 566 of the receiver 510. The flange form utilized in accordance with the present invention may take a variety of forms, including those described in Applicant's U.S. Pat. No. 6,726,689, which is incorporated herein by reference. It is also foreseen that according to the invention the closure structure guide and advancement structure could alternatively be a buttress thread, a square thread, a reverse angle thread or other thread like or non-thread like helically wound advancement structure for operably guiding under rotation and advancing the closure structure 518 downward between the arms 560 and having such a nature as to resist splaying of the arms 560 when the closure structure 518 is advanced therebetween. The illustrated closure structure 518 also includes a top surface 686 with an internal drive 687 in the form of an aperture that is illustrated as a star-shaped internal drive, but may be, for example, a hex-shaped drive or other internal drives, including, but not limited to slotted, tri-wing, spanner, two or more apertures of various shapes, and the like. A driving tool (not shown) sized and shaped for engagement with the internal drive 687 is used for both rotatable engagement and, if needed, disengagement of the closure 618 from the receiver arms 560. It is also foreseen that the closure structure 518 may alternatively include a break-off head designed to allow such a head to break from a base of the closure at a preselected torque, for example, 70 to 140 inch pounds. Such a closure structure would also include a base having an internal drive to be used for closure removal. A bottom surface 688 of the closure top 10 is planar, but may include a point, points, a rim or roughening for engagement with the rod 521. Furthermore, in some embodiments, the closure top may include an extended base or knob for pressing into a deformable rod or compressing a cord against the insert seating surface. Such an extension or knob would be sized and shaped to extend into the channel 614 of the insert 516 and also clear the walls defining the channel 614 so that a portion of the closure top would abut against the insert 516, locking the polyaxial mechanism of the bone screw.

The closure top 518 may further include a cannulation through bore extending along a central axis thereof and through a surface of the drive 687 and the bottom surface 688. Such a through bore provides a passage through the closure 518 interior for a length of wire (not shown) inserted therein to provide a guide for insertion of the closure top into the receiver arms 560.

With particular reference to FIGS. 44 and 52, at the factory, the illustrated shank upper portion 508 is bottom loaded into the receiver 510 and the retainer 512 is top loaded into the receiver 510 in the same manner as previously described herein with respect to the retainer 12 and the receiver 10 of the assembly 1. The shank upper portion 508 is aligned with respect to the retainer 512 such that the projections 538 are passed through the retainer 512 central bore 581 between each of the shelves 588, with each of the outer cylindrical surfaces 548 in slidable engagement with the inner cylindrical surface 586 of the retainer 512. Once the bottom surfaces 547 of the projections 538 are disposed above and spaced from the seating surface 590 of the shelves 588, the shank 504 and/or the retainer 512 is rotated about the receiver 510 until the projections 538 are axially aligned with the shelves 588. It is foreseen that in some embodiments, the shank is top loaded. Upon alignment, the shank upper portion 508 and the retainer 512 are then moved toward one another along the receiver 510 axis until the surfaces 547 fully frictionally engage the shelf surfaces 590. Then, small crimp portions 598 are cut into the surface 582 of the retainer 512. It is foreseen that the crimp portions 598 can be pre-cut. The portions 598 remain integral with the retainer 512 but are also movable and pressable in a radial direction into the retainer bore 581. Such cutting may be performed by an EDM (electrical discharge machining) process. Such a process allows for cut patterns on the retainer 512 by eroding material in the path of electrical discharges that form and arc between an electrode tool and the retainer, producing efficient, accurate, and in the present case, desirable, partial cuts in the retainer 512 that are then pressed toward the shank upper portion 508 at locations adjacent projection side surfaces 550 and 551, thereby crimping the retainer 512 towards and against the shank upper portion 508, fixing the retainer 512 to the shank 504 at the upper portion 508 after the shank and retainer have come into proper alignment. At this point there is no substantial outward or downward pressure on the retainer 512 and so the retainer 512 is easily rotatable and pivotable along with the shank 506 within the seating surface portion 576 of the chamber 572 and such movement is of a ball and socket type wherein the degree of angulation or pivot is only restricted by engagement of the shank neck with the neck of the receiver 510.

Then, the insert 516 is inserted into the receiver u-shaped channel with the arms 612 aligned in the channel between the guide and advancement structures 566. The insert 516 is then moved downwardly toward the cavity 572. Once the arms 612 are located generally below the guide and advancement structure 566, the insert 516 is rotated about the axis of the receiver 510. The arms 612 fit within the cylindrical walls 574 of the cavity 572 above the spherical seat 576. Once the arms 612 are located directly below the guide and advancement structures 566, rotation is ceased and a tool (not shown) is directed into apertures 570 to press the thin walls of the receiver 510 into the recesses 624 of the insert 516. The insert 516 is now locked into place inside the receiver 510 with the guide and advancement structures 566 prohibiting upward movement of the insert upwardly along and out of the receiver arms 560. As illustrated in FIG. 52, the insert 516 seats on the shank upper portion 508 with the surface 636 in sliding engagement with the surface 537 and possibly portions of projection upper surfaces 546. A run-out or relief located below the guide and advancement structures 566 is sized and shaped to allow for some upward and downward movement of the insert 516 toward and away from the shank upper portion 508 such that the shank 506 is freely pivotable with respect to the receiver 510 until the closure structure 518 presses on the insert 516 that in turn presses upon the upper portion 508 into locking frictional engagement with the receiver 510 at the surfaces 597 and 576.

The resulting bone screw is then normally screwed into a bone, such as vertebra as previously described with respect to the assembly 1, with a driving tool (not shown) engaging the internal drive feature 540 of the bone screw shank 504. At this time, the retainer 512 outer curved surface 597 is in sliding, pivotal engagement with the inner seating surface 576 of the receiver 510, allowing for a wide range of pivotal movement between the shank 504 and the receiver 510. Thus, both the shank 504 and the retainer 512 are in rotatable and swivelable engagement with the receiver 510, while the shank upper portion 508 and the lower aperture or neck of the receiver 510 cooperate to maintain the shank body 506 in swivelable relation with the receiver 510. Only the retainer 512 is in slidable engagement with the receiver inner spherical seating surface 576. The shank upper end surface 537 and the shank body 506 are in spaced relation with the receiver 510 seating surface 576. The shank body 506 can be rotated through a substantial angular rotation relative to the receiver 510, both from side to side and from front to rear so as to substantially provide a universal or ball joint.

The rod 521 is eventually positioned within the U-shaped channel 614, as is seen in FIG. 52, and the closure top 518 is then inserted into and advanced between the receiver arms 560 so as to bias or push the rod 521 downwardly toward the insert seating surface 616. A driving tool (not shown) is inserted into the drive 687 to rotate and drive the closure top 518 into the receiver 510. The shank dome 537 is engaged by the insert 516 and pushed downwardly when the closure top 518 pushes downwardly on the rod 521, locking the rod and the bone screw polyaxial mechanism in a desired position.

If removal of the assembly 501 is necessary, the assembly 501 can be disassembled by using a driving tool mating with the closure top aperture 687 to rotate the closure top 518 and reverse the advancement thereof in the receiver 510. Then, disassembly of the remainder of the assembly 501 may be accomplished in reverse mode in comparison to the procedure described above for assembly.

With reference to FIGS. 53 and 54, an alternative shank 504′ and cooperating retainer 512′ are illustrated. The shank 504′ and the retainer 512′ are identical to the respective shank 504 and retainer 512 described previously herein prior to cutting of crimp portion 598′. Therefore identical numerals have been used to identify the elements thereof with the addition of a “′” (prime) notation to note that the resulting assembly is different from the assembly 501, but only with respect to the size of the cut-out crimped portions 598′ as compared to the small crimped portions 598 of the assembly 501. In the embodiment shown in FIGS. 53 and 54, cuts are made to the retainer 512′ surface 582′ that extend an entire distance between shank projection end surfaces 550′ and 551′. The cut-out portions 598′ remain integral with the retainer 512′ and are movable portions 598′ created by an EDM cutting process. Each of three portions 598′ is moved and pressed against the shank upper portion 508′ upper cylindrical body 534′ between each of the projections 538′ to securely fix the retainer 512′ to the shank upper portion 508′ at a location spaced from the shank upper domed surface 537′. The domed surface 537′ slidingly mates with the lower pressure insert 516 as previously discussed herein with respect to the upper surface 537 of the pressure insert 516.

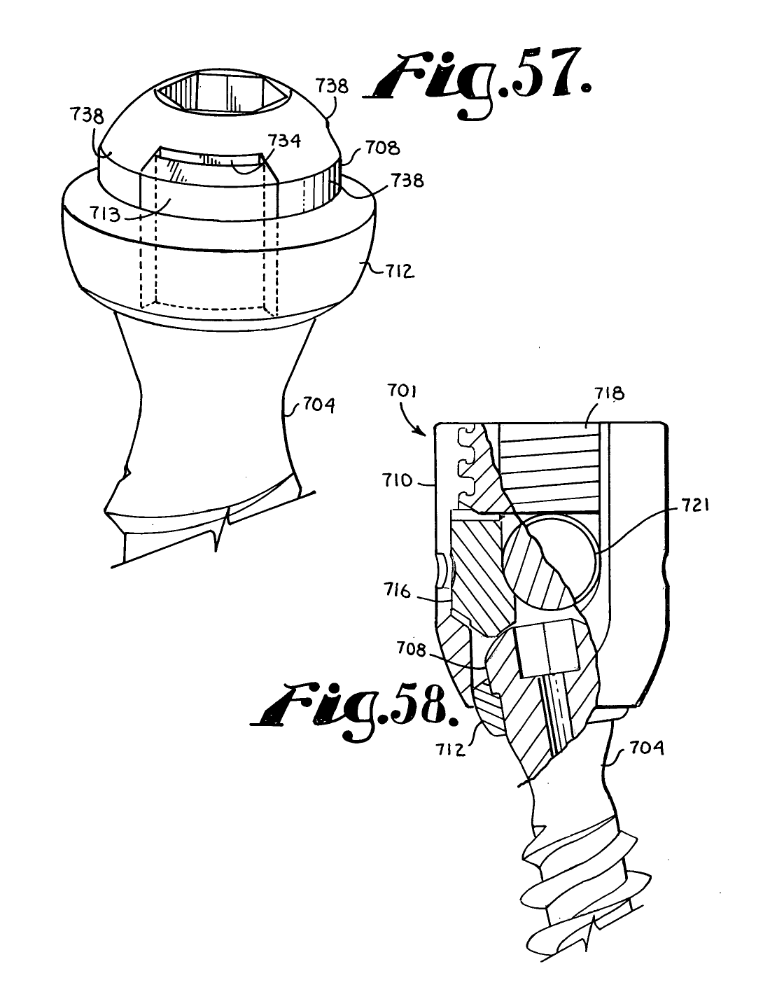

With reference to FIGS. 55-58, the reference number 701 generally represents another alternative embodiment of a polyaxial bone screw apparatus or assembly according to the present invention. The assembly 701 is identical to the assembly 501 with the exception that inserts or wedges 713 are used in lieu of the crimp cut-outs 598. Thus, the assembly 701 includes a shank 704 having an upper portion 708 with three projections 738, a receiver 710, a retainer 712 having three shelves 788, a lower pressure insert 716, and a closure 718 identical or substantially similar to the respective shank 504 with upper portion 508 and projections 538, receiver 510, retainer 512 with three shelves 588, lower pressure insert 516 and closure 518 previously described herein with respect to the assembly 501. As best shown in FIGS. 56 and 57, each of the three wedges 713 is sized and shaped to be closely frictionally received between neighboring projections 738 as well as between neighboring shelves 788 and closely, frictionally sandwiched between the shank upper portion 708 at a cylindrical body surface 734 and a surface 781 defining the retainer inner bore.

With reference to FIG. 59, an alternative retainer 712′ is illustrated. The retainer 712′ is for the most part identical to the retainers previously described herein, in particular, the retainers 712 and 512 and may be utilized in assemblies substantially similar to the assemblies 701 and 501 previously described herein as well as other assemblies previously described herein that do not include a compression insert. Therefore, identical numerals have been used to identify the elements thereof with the addition of a “′” (prime) notation to note that the resulting assembly is different from the retainer 712 of the assembly 701, but only with respect to the size of inner shelves 788′. In the embodiment shown in FIG. 59, the shelves 788′ extend all the way to a top surface 782′ of the retainer 712′, each projection having a top surface 790′ that is adjacent the retainer top surface 782′. Thus, the retainer 712′ cooperates with a shank (not shown) according to the invention substantially similar to the shank 704, but having shorter projections that engage the shelves 788′ at the surfaces 790′ located at the top of the retainer 712′ rather than along an interior surface thereof. The retainer 712′ is then fixed to the shank by spot welds, wedges similar to the wedges 713, or with other methods and crimping structure previously described herein.

With reference to FIG. 60, the reference number 712″ represents another alternative embodiment of a retainer for use in a polyaxial bone screw apparatus or assembly according to the present invention. The retainer 712″ is identical to the previously described retainer 712 with the exception that the retainer 712″ is an open structure, having a through slit running from a top to a bottom thereof, defined by facing surfaces 798″ and 799″ that are spaced from one another as shown by the gap G. Such an open retainer 712″ may be uploaded into a receiver according to the invention, for example, the receiver 510, by compressing the retainer 712″ by pinching the surfaces 798″ and 799″ toward one another and inserting the retainer 712″ into the receiver base 558 through the lower opening thereof and directly into the inner cavity 572. The resilient retainer 712″ then returns to a neutral and open position once inside the cavity 572. Thereafter, a shank according to the invention, for example the shank 504, may be uploaded or downloaded into the receiver and then frictionally engaged to the retainer 712″ at the shelves 788″ as described previously with respect to other embodiments of the invention.

It is to be understood that while certain forms of the present invention have been illustrated and described herein, it is not to be limited to the specific forms or arrangement of parts described and shown.

What is claimed and desired to be secured by Letters Patent is as follows:

1. A polyaxial bone anchor comprising:
a) a shank having a body for fixation to a bone and an integral upper portion, the upper portion having an upper end surface spaced from at least one laterally directed projection, the projection having a bottom surface, the upper end surface engageable with one of a rod and an insert;
b) a receiver having a top portion and a base, the receiver top portion defining an open channel, the base having a seating surface partially defining a cavity, the channel communicating with the cavity, the cavity communicating with an external of the base through an opening sized and shaped to receive the shank body therethrough; and
c) a retainer having an external surface, a top surface and a central bore with an internal surface defining the central bore having at least one shelf for engaging the bottom surface of the projection of the shank upper portion, the shank upper portion and the retainer being in fixed relation to one another, both the upper portion and the retainer being in swivelable relation within the receiver, providing selective angular positioning of the shank with respect to the receiver, the retainer external surface being in slidable engagement with the receiver seating surface, the retainer top surface being in spaced relation with the upper end surface of the shank.

2. The bone anchor of claim 1 further comprising a compression insert disposed in the receiver, the compression insert having a mating surface exclusively frictionally engageable with the shank upper end surface.

3. The bone anchor of claim 2 wherein the receiver has a projected wall surface engaging the compression insert.

4. The bone anchor of claim 3 wherein the projected wall surface prevents rotational movement of the compression insert.

5. The bone anchor of claim 1 wherein the shelf is spaced beneath the retainer top surface.

6. The bone anchor of claim 1 wherein the shelf is adjacent the retainer top surface.

7. The bone anchor of claim 1 wherein the projection is press fit against the shelf.

8. The bone anchor of claim 1 wherein the retainer is top loadable into the receiver.

9. The bone anchor of claim 1 wherein the retainer is bottom loadable into the receiver.

10. The bone anchor of claim 1 wherein the retainer has a through slit running from the external surface to the internal surface and from the top surface to a base surface thereof.

11. The bone anchor of claim 10 wherein the slit runs in a direction substantially perpendicular to the base surface.

12. The bone anchor of claim 1 wherein the receiver base opening is sized and shaped to receive the shank upper portion therethrough.

13. The bone anchor of claim 1 wherein the retainer includes at least one deformable wall portion, the deformable wall portion being pressed against the projection.

14. The bone anchor of claim 13 wherein the projection includes an aperture, the deformable wall portion being pressed into the aperture.

15. The bone anchor of claim 13 wherein the deformable wall portion is located at the retainer internal surface.

16. The bone anchor of claim 13 wherein the deformable wall portion is located at the retainer top surface.

17. The bone anchor of claim 13 wherein the deformable wall portion is a spring tab.

18. The bone anchor of claim 13 wherein the deformable wall portion is a thin, pressable wall.

19. The bone anchor of claim 13 wherein the deformable wall portion is a pre-cut crimp edge.

20. The bone anchor of claim 19 wherein the projection is a first projection and further comprising a second projection and the pre-cut crimp edge extends between the first and second projections.

21. The bone anchor of claim 1 further comprising a pin and wherein the retainer includes at least one through aperture running between the external and the internal surface and the projection includes a receiving aperture, the through aperture and the receiving aperture being in alignment, the pin extending through the through aperture and closely, frictionally received by the receiving aperture.

22. The bone anchor of claim 1 wherein the at least one projection is a first projection and further comprising a second projection and a wedge, the wedge being press fit between the first and second projections and against the retainer internal surface.

23. A polyaxial bone anchor comprising:
a) a shank having a body for fixation to a bone and an integral upper portion, the upper portion having an upper end surface and at least one laterally directed projection having a bottom surface;
b) a receiver having a top portion and a base, the receiver top portion defining an open channel, the base having a seating surface partially defining a cavity, the channel communicating with the cavity, the cavity communicating with an external of the base through an opening sized and shaped to receive at least the shank body therethrough;
c) a compression insert disposed in the receiver, the insert having a mating surface exclusively frictionally engageable with the upper end surface of the shank upper portion;
d) a retainer having an external surface and a central bore with an internal surface defining the central bore having at least one substantially planar shelf for frictionally engaging the shank projection bottom surface, the shank upper portion and the retainer being in fixed relation to one another, both the upper portion and the retainer being in swivelable relation within the receiver, providing selective angular positioning of the shank with respect to the receiver, the retainer external surface being in slidable engagement with the receiver seating surface, the retainer being substantially spaced from the compression insert at any and all angular positions of the shank with respect to the receiver; and
e) a holding structure for retaining fixed relation between the shank upper portion and the retainer.

24. The bone anchor of claim 23 wherein the holding structure is a deformable wall located on the retainer.

25. The bone anchor of claim 23 wherein the holding structure is a pin extending through at least a portion of each of the retainer and the projection.

26. The bone anchor of claim 23 wherein the holding structure is a spring tab.

27. The bone anchor of claim 23 wherein the holding structure is a cut and crimp edge portion located at a top surface of the retainer.

28. The bone anchor of claim 23 wherein the shank is cannulated.

29. The bone anchor of claim 23 wherein the shank has an internal drive feature.

30. A polyaxial bone screw assembly comprising:
a) a receiver having a channel adapted to receive an elongate longitudinal member and having a lower opening;
b) a shank having an axis of rotation, a lower portion adapted to be implanted in a bone of a patient and an upper portion, the shank upper portion having at least one laterally directed projection with a first seating surface substantially perpendicular to the axis of rotation, the shank being sized and shaped to be positioned in the receiver so as to extend through the receiver lower opening; and
c) a retaining structure non-integral with the shank and loaded into the receiver separately from the shank, the retaining structure mating with and being secured to the shank by downward positioning of the laterally directed projection onto a second seating surface in the retaining structure, the second seating surface being substantially planar, the first and second seating surfaces in frictional engagement so as to capture the retaining structure with the shank upper portion in the receiver so as to allow polyaxial movement of the shank relative to the receiver.

31. The bone screw assembly of claim 30 further comprising a compression insert directly and frictionally engaging the shank upper portion and in spaced relation with the retaining structure.

32. The bone screw assembly of claim 30 further comprising a holding structure for securing the retaining structure to the shank upper portion.

33. The bone screw assembly of claim 32 wherein the holding structure is a deformable wall located on the retaining structure.

34. The bone screw assembly of claim 32 wherein the holding structure is a pin extending through at least a portion of each of the retaining structure and the projection.

35. The bone screw assembly of claim 32 wherein the holding structure is a spring tab.

36. The bone screw assembly of claim 32 wherein the holding structure is a crimped edge portion of the retaining structure.
